(19) United States
(12) Patent Application Publication
(10) Pub. No.: US 2014/0287004 A1
(43) Pub. Date: Sep. 25, 2014

(54) Use of an Omega-3 Lipid-Based Emulsion Following Ischemic Injury to Provide Protection and Recovery in Human Organs

(71) Applicant: Richard J. Deckelbaum, Hastings on Hudson, NY (US)

(72) Inventor: Richard J. Deckelbaum, Hastings on Hudson, NY (US)

(73) Assignee: The Trustees of Columbia University in the City of New York, New York, NY (US)

(21) Appl. No.: 14/299,440

(22) Filed: Jun. 9, 2014

Related U.S. Application Data
(63) Continuation-in-part of application No. 13/336,290, filed on Dec. 23, 2011.
Application No. 13/336,290 is a continuation-in-part of application No. PCT/US14/17523, filed on Feb. 20, 2014.
Application No. PCT/US14/17523 is a continuation-in-part of application No. PCT/US14/32279, filed on Mar. 28, 2014.
(60) Provisional application No. 61/806,391, filed on Mar. 28, 2013.
(60) Provisional application No. 61/767,248, filed on Feb. 20, 2013.
(60) Provisional application No. 60/799,677, filed on May 12, 2006.
(60) Provisional application No. 60/735,862, filed on Nov. 14, 2005.

(51) Int. Cl. A61K 31/202; A61K 9/107

(52) U.S. Cl. CPC A61K 31/202; A61K 9/1075
USPC 424/400; 514/560; 424/523

(57) ABSTRACT

The present invention provides methods of limiting cell death or damage or reperfusion damage resulting from hypoxic-ischemia, comprising administering an omega-3 triglyceride emulsion after a hypoxic-ischemia insult. The omega-3 triglyceride emulsion preferably comprises from about 7% to 35% omega-3 oil by weight in grams per 100 ml of emulsion; the omega-3 oil comprises about 20% to 100% triglyceride by weight per total weight of the omega-3 oil and about 20% wt.-% to 100% of the acyl-groups of the omega-3 triglycerides consist of DHA; the omega-3 oil comprises less than 10% omega-6 fatty acids; and the mean diameter of lipid droplets in the emulsion is less than about 5 microns.

### CROSS-REFERENCE TO RELATED APPLICATIONS

This application is a continuation-in-part application of PCT/US 14/17523, filed on Feb. 20, 2014 designating the United States, and entitled “Use of an Omega-3 Lipid-Based Emulsion Following Ischemic Injury to Provide Protection and Recovery in Human Organs,” and claims priority to U.S. provisional application Ser. No. 61/767,248, filed on Feb. 20, 2013, entitled “Omega-3 Triglyceride-DHA Emulsions for the Treatment of Hypoxic-ischemic Injuries.” This application is also a continuation-in-part application of PCT/US2014/32279, filed Mar. 28, 2014, entitled “Reperfusion with Omega-3 Glycerides Promotes Donor Organ Protection for Transplantation,” and claims priority to U.S. Provisional Application Ser. No. 61/806,391, filed Mar. 28, 2013, entitled “Reperfusion with Omega-3 Glycerides Promotes Donor Organ Protection for Transplantation.” This application is also a continuation-in-part application of U.S. application Ser. No. 13/336,290, filed Dec. 23, 2011, entitled “Use of an Omega-3 Lipid-Based Emulsion Following Ischemic Injury to Provide Protection and Recovery in Human Organs,” and claims priority to U.S. application Ser. No. 11/558,568, filed on Nov. 10, 2006, entitled “Use of an Omega-3 Lipid-Based Emulsion Following Ischemic Injury to Provide Protection and Recovery in Human Organs,” and claims priority to U.S. provisional application Ser. No. 60/799,677, filed on May 12, 2006, entitled “Use of an Omega-3 Lipid-Based Emulsion Following Ischemic Injury to Provide Protection and Recovery in Human Organs,” and U.S. provisional application Ser. No. 60/735,862, filed on Nov. 14, 2005, entitled “Omega-3 Long Chain Polyunsaturated Fatty Acids as a Neuroprotective Agent Following an Ischemic Insult,” all of which are herein incorporated in their entirety under 35 U.S.C. under 35 U.S.C. §120.

### BACKGROUND

Cerebral hypoxia-ischemia (stroke) is a major cause of morbidity and mortality through all stages of the life cycle, including for infants born prematurely, for children in intensive care units, and for elderly with cerebral vascular accidents. Infants and children who survive hypoxic-ischemic encephalopathy demonstrate lifelong neurologic handicaps, including cerebral palsy, mental retardation, epilepsy, and learning disabilities. Vannucci, R. C. (2000) “Hypoxic-ischemic encephalopathy,” American Journal of Perinatology 17(3): 113-120.

Cerebral hypoxia-ischemia commonly occurs in critically ill children, most notably in association with cardiopulmonary arrest. Lipid emulsions are commonly used in pediatric intensive care and are an important source of calories in these critically ill children. Most commercially available emulsions are formed from soybean oil, which has high concentrations of omega-6 (n-6) fatty acids. Lipid emulsions rich in omega-3 (n-3) fatty acids such as α-linolenic acid, eicosapentaenoic acid (EPA), and docosahexaenoic acid (DHA) are derived from fish oils, and are not yet widely available for clinical use. However, increased intakes of omega-3 oils from diet or supplements have been shown to have beneficial effects in neurologic diseases such as epilepsy, depression, and behavioral disorders. Most studies support a neuroprotective effect due to dietary administration leading to altered membrane lipid composition. In one study, intravenous α-linolenic acid given before and/or after neurologic insult was protective in two animal models, kainate-induced seizures and global ischemia via four vessel occlusion in adult Sprague-Dawley rats. Lauritzen, I., et al. (2000), “Polyunsaturated fatty acids are potent neuroprotectors,” The EMBO Journal 19(8):1784-93. Stroke is the third to fourth most common cause of death in adults and carries huge costs in terms of not just mortality but also care for the consequences of stroke in survivors.

Acute myocardial infarction (MI) is one of the major causes of death despite substantial advancement in diagnosis and therapy in recent decades. Coronary heart disease caused 1 of every 6 deaths in the United States in 2009. Hypoxia, energy depletion, and ion homeostasis alterations characterize ischemic conditions. Duration of ischemia has been predictive of severity of myocardial injury. The efficacy of reperfusion, stage that occurs immediately after ischemia, is also a key and crucial factor. If not effective, reperfusion may induce additional and pronounced impairment. Myocardial ischemia/reperfusion (I/R) injury provokes irreversible metabolic and structural changes. There remains a need for methods to acutely protect the heart, brain, and other organs and tissues against damage after an initial ischemic insult. The present invention fulfills this need.

### SUMMARY OF THE INVENTION

The present invention provides triglyceride omega-3 lipid-based oil-in-water emulsions suitable for administration to a patient. These emulsions comprise at least 7% to 35% omega-3 oil and less than 10% omega-6 oil by weight in grams per 100 ml of emulsion. The omega-3 oil comprises at least 20% triglyceride by weight per total weight of the omega-3 oil, and at least 20% wt.-% of the acyl-groups of the omega-3 triglycerides consist of DHA. The omega-3 oil comprises less than 10% omega-6 fatty acids, and the mean diameter of lipid droplets in the emulsion is less than about 5 microns. The lipid droplets are less than about 1 micron diameter or from about 100 to about 500 nm diameter. In certain embodiments, the omega-3 oil is fish oil, synthetic omega-3 oil, or a combination thereof.

In certain embodiments, from 20 wt.-% to 50 wt.-% of the acyl-groups of the omega-3 triglycerides consist of DHA. In other embodiments, from 50 wt.-% to 75 wt.-% of the acyl-groups of the omega-3 triglycerides consist of DHA. In yet other embodiments, from 75 to 90 wt.-% of the acyl-groups of the omega-3 triglycerides consist of DHA. In certain embodiments, from 90 to 95 wt.-% of the acyl-groups of the omega-3 triglycerides consist of DHA. And in other embodiments, from 95 to 100 wt.-% of the acyl-groups of the omega-3 triglycerides consist of DHA.

Methods are provided in certain embodiments for identifying a subject who has undergone hypoxia-ischemia, and then administering to the subject a pharmaceutical composition comprising a therapeutically effective amount the triglyceride omega-3 lipid-based oil-in-water emulsion of claim 1 to reduce reperfusion damage caused by the hypoxia-ischemia. The hypoxia-ischemia causes cerebral hypoxia-ischemia, and the therapeutically effective amount of the triglyceride omega-3 lipid-based oil-in-water emulsion is administered within 20 minutes to 6 hours after the cerebral hypoxia-ischemia to reperfusion damage caused by the hypoxia-ischemia. The cerebral hypoxia-ischemia causes a stroke. In certain embodiments, the therapeutically effective amount is from about 0.05 g/kg/administration to about 4 g/kg/administration. In other embodiments, the hypoxia-ischemia and the reperfusion damage caused by the hypoxia-ischemia occurs in an organ selected from the group consisting of brain, heart, kidney, spinal cord, large or small intestine, pancreas and lung. Hypoxia-ischemia and the reperfusion damage caused by the hypoxia-ischemia in other embodiments causes a myocardial infarction or a cerebral infarction.

In other embodiments, methods are provided for reducing cell death or damage in an organ or tissue caused by hypoxia-ischemia or reperfusion damage caused by the hypoxia-ischemia comprising administering to a patient in need thereof a pharmaceutical composition comprising a therapeutically effective amount the triglyceride omega-3 lipid-based oil-in-water emulsion. In certain embodiments, the hypoxia-ischemia or reperfusion damage caused by the hypoxia-ischemia is caused by organ transplantation. In other embodiments, the cell death or cell damage occurs in an organ selected from the group consisting of brain, heart, kidney, spinal cord, large or small intestine, lung, liver, and pancreas. In certain embodiments, the hypoxia-ischemia causes a stroke. In still other embodiments, the hypoxia-ischemia causes a myocardial infarction. The therapeutically effective amount is from about 0.05 g/kg/administration to about 4 g/kg/administration.

In other embodiments, methods are provided for identifying a subject who is at risk of having a cerebral hypoxia-ischemia, and administering to the subject a pharmaceutical composition comprising a therapeutically effective amount of the triglyceride omega-3 lipid-based oil-in-water emulsion of claim 1, thereby reducing the risk of the subject developing reperfusion damage caused by the hypoxia-ischemia.

In certain embodiments, methods are provided for identifying a subject who has inflammation in an organ, and administering to the subject a pharmaceutical composition comprising a therapeutically effective amount of the triglyceride omega-3 lipid-based oil-in-water emulsion of claim 1, thereby reducing the inflammation in the subject.

In other embodiments, methods are provided for administering to a subject an amount of a pharmaceutical composition comprising the triglyceride omega-3 lipid-based oil-in-water emulsion, to reduce production of reactive oxygen species in the blood or in an organ in the subject. The organ is selected from the group consisting of brain, heart, kidney, spinal cord, large or small intestine, lung, liver, and pancreas.

Methods are provided for reducing adverse cytokine production in a subject, comprising administering to a subject in need thereof a therapeutically effective amount of a pharmaceutical composition comprising the triglyceride omega-3 lipid-based oil-in-water emulsion. The emulsion comprises at least 20% to 30% omega-3 oil by weight in grams per 100 ml of emulsion. In certain embodiments, the emulsion comprises at least 30% to 35% omega-3 oil by weight in grams per 100 ml of emulsion. In other embodiments, the omega-3 oil comprises at least 30% to 40% triglyceride by weight per total weight of the omega-3 oil. In other embodiments, the omega-3 oil comprises at least 40% to 50% triglyceride by weight per total weight of the omega-3 oil. In still other embodiments, the omega-3 oil comprises at least 50% to 75% triglyceride by weight per total weight of the omega-3 oil. In certain embodiments, the omega-3 oil comprises at least 75% to 100% triglyceride by weight per total weight of the omega-3 oil. In other embodiments, the emulsion comprises less than 5% omega-6 fatty acid or less than 1% omega-6 fatty acid. In other embodiments, the emulsion comprises at least 35% omega-3 oil by weight in grams per 100 ml of emulsion and less than 3% omega-6 oil.

In certain embodiments, methods are provided for identifying a subject who has inflammation in an organ or a tissue, and administering to the subject a pharmaceutical composition comprising a therapeutically effective amount the triglyceride omega-3 lipid-based oil-in-water emulsion thereby reducing the inflammation in the subject.

In other embodiments, methods are provided for reducing cell death or damage or hypoxia/ischemia in an organ or tissue comprising administering to a an organ donor a pharmaceutical composition comprising a therapeutically effective amount the triglyceride omega-3 lipid-based oil-in-water emulsion prior to harvesting the organ or tissue from the organ donor.

In still other embodiments, methods are provided for reducing cell death or damage or hypoxia/ischemia in an organ or tissue to be transplanted into an organ or tissue recipient, comprising administering to the recipient a pharmaceutical composition comprising a therapeutically effective amount the triglyceride omega-3 lipid-based oil-in-water emulsion of prior to surgically implanting the organ or tissue in the recipient.

### BRIEF DESCRIPTION OF THE DRAWINGS

The present invention is illustrated by way of example, and not by way of limitation, in the figures.

FIG. 1A-1C. FIG. 1A gives the mean damage score for the brain of rats given 20% fish oil emulsion—50 mg 20% omega-3 triglyceride lipid-based emulsion (0.25 cc), a 20% long chain omega-3 triglyceride-based formula having >45% of total omega-3 fatty acid as eicosapentaenoic acid (EPA) and docosahexaenoic acid (DHA)—or water (controls) as a neuroprotective agent both prior and after 60 minutes of cerebral hypoxic-ischemia. The damage was determined 72 hours after 60 min of hypoxia-ischemia (H/I). FIG. 1B and FIG. 1C are photographs showing the omega-3 emulsion-treated vs. control brain tissue after 60 min of H/I.

FIG. 2A-2C. FIG. 2A provides the results of an experiment where rats were given an omega-3 triglyceride lipid-based emulsion-50 mg 20% omega-3 triglyceride lipid-based emulsion (0.25 cc), a 20% long chain omega-3 triglyceride-based formula having >45% of total omega-3 fatty acid as eicosapentaenoic acid (EPA) and docosahexaenoic acid (DHA)—or water (controls) as a neuroprotective both prior and after 65 minutes of hypoxic-ischemia. These were enteral experiments using 1-1.5 mg/g body weight. The damage was determined 72 hours after 60 min of H/I. FIG. 2B and FIG. 2C are photographs showing the omega-3 emulsion-treated vs. control brain tissue 72 hours after 60 min of H/I.

FIG. 3A-3B show the neuroprotective effect of administering the omega-3 triglyceride-50 mg 20% omega-3 triglyceride lipid-based emulsion (0.25 cc), a 20% long chain omega-3 triglyceride-based formula having >45% of total omega-3 fatty acid as eicosapentaenoic acid (EPA) and docosahexaenoic acid (DHA)—just before hypoxic/ischemic insult on the size of brain infarcts as determined by TTC staining.

FIG. 4 shows the results of four separate experiments where rats were given an omega-3 triglyceride lipid-based emulsion (50 mg 20% omega-3 triglyceride lipid-based emulsion (0.25 cc) (a 20% long chain omega-3 triglyceride-based formula having >45% of total omega-3 fatty acid as eicosapentaenoic acid (EPA) and docosahexaenoic acid (DHA)) after 60 minutes of hypoxic-ischemia. 40% of the treated animals had no brain damage and another 40% had less damage than the mean of control animals as assessed by TTC staining.

FIG. 5A-5B are graphs that illustrate blood TG and glucose levels after TG emulsion injections. FIG. 5A is a graph that illustrates total plasma TG concentrations (mg/dL) in non-fasting neonatal mice (p10), acutely injected intraperitoneally (“i.p.”) with saline or n-3 TG emulsion (0.75 g n-3 TG/kg body weight). *p<0.05 (n=3-8 in each group). Each data point represents the mean±SEM of 3 separate experiments. FIG. 5B is a bar graph that illustrates plasma glucose concentrations (mg/dL) in non-fasting mice (p10) in post-H/I treatment of n-3 TG or n-6 TG or vehicle (saline) comparing to the time between before H/I and after H/I. **p<0.001 (n=5-9 in each group).

FIG. 6A-6C are graphs that illustrate TTC stained coronal sections of mouse brain and quantification of injury after H/I. FIG. 6A is a micrograph that illustrates TTC-stained coronal sections of representative mouse brains from saline treated, n-3 TG treated and n-6 TG treated. The top panel shows images of coronal mouse brain that are sliced and then stained with TTC (gray for living tissue and white for the infarcted tissue), and the lower panel shows the infarcted areas that are traced in black for quantification. FIG. 6B is a bar graph that illustrates percent of cerebral infarct volume from pre-H/I mice treated with n-3 TG emulsion (n=28) or n-6 TG emulsion (n=10) or saline control (n=27). FIG. 6C is a bar graph that illustrates percent of cerebral infarct volume after H/I in the post-H/I treatment protocol in mice treated with n-3 TG emulsion (n=18) or saline control (n=18). Each bar represents the mean±SEM of 5-7 independent experiments.

FIG. 7 is a graph that illustrates n-3 TG injection and cerebral blood flow after H/I. Cerebral blood flow (CBF) was measured by laser Doppler flowmetry (LDF) in neonatal mice after carotid artery ligation. Relative CBF was measured every two minutes during hypoxia in ipsilateral (right) hemispheres using a laser Doppler flowmeter. Changes in CBF in response to hypoxia were recorded for 20 min and expressed as percentage of the pre-hypoxia level for n-3 TG treated (n=3) and saline treated (n=5) neonatal mice.

FIG. 8 are bar graphs that illustrate acute n-3 TG (1-1.5 mg/g body weight EPA+DHA were ˜48% of the FAs by weight) neuroprotection in hypoxia/ischemia as quantified for the (left) juvenile rat, (middle) adult mouse, and (right) neonatal mouse. % infarct volume was measured and it was determined that the n3-TG treated group on right in each bar graph had significantly less damage as compared to the saline treated controls on the left of each bar graph.

FIG. 9 is a graph that illustrates attenuation of brain injury by n-3 TG after H/I. After injection of n-3 TG (1-1.5 mg/g body weight EPA+DHA were ˜48% of the FAs by weight), different regions of the brain are markedly protected from stroke injury after H/I.

FIG. 10A-10B are graphs that illustrate acute n-3 TG injection decreases brain Ca²⁺ induced opening of mitochondrial permeability transition pores (mPTP) after H/I. After H/I and after n-3 TG (1-1.5 mg/g body weight EPA+DHA were ˜48% of the FAs by weight) injection, mitochondrial function is maintained.

FIG. 11A-11B are graphs that illustrate n-3 DG decreases brain injury and infarct volume in H/I neonatal mice.

FIG. 12 is a bar graph that illustrates DHA content in brain mitochondria. At 4 hours after injection of the n-3 TG (0.75 mg/g body weight) emulsion after stroke, DHA content in brain mitochondria is increased and that this increase likely contributes to the beneficial effects of DHA. Note that EPA content was not increased in brain mitochondria (data not shown).

FIG. 13 is a bar graph that illustrates infarct volume at 24 hours post H/I in mice post-treated with NS (vehicle) or NPD1 (20 ng). NPD1 is a catabolic product of DHA.

FIG. 14A-14B are graphs that illustrate the effect of Tri-DHA versus Tri-EPA on cerebral infarct volume after H/I. FIG. 14A is a bar graph that illustrates mice subjected to 15 min ischemia followed by 24-hr reperfusion and received 2 i.p. administrations (immediately after ischemia and 1 hr. of reperfusion) at 2 doses (0.1 g n-3 TG/kg and 0.375 g n-3 TG/kg). Each bar represents the mean±SEM of 5-7 independent experiments performed using the same H/I model. FIG. 14B are micrographs that illustrate TTC-stained coronal sections of representative mouse brains from saline treated, 0.1 g Tri-DHA, 0.375 g Tri-DHA, 0.1 g Tri-EPA and 0.375 g Tri-EPA. *p<0.05 compared to other groups except 0.1 g Tri-DHA/kg. **p<0.05 compared to other groups except 0.375 g Tri-DHA/kg and 0.375 g Tri-EPA/kg.

FIG. 15 is a bar graph that illustrates the effects of delayed treatment with Tri-DHA on cerebral infarct volume after H/I. Mice were subjected to 15-min ischemia followed by 24-hr reperfusion and received 2 i.p. administrations at four-time points (immediate [0,1 hr], delayed 1-hr [1,2 hr], or 2-hr [2,3 hr] or 4-hr [4,5 hr] treatments). Each bar represents the mean±SEM of 5-7 independent experiments. *p<0.05; **p<0.001 vs. saline control (n=10-20 in each group).

FIG. 16 is a bar graph that illustrates the long-term effect of Tri-DHA on cerebral tissue death at 8 wk. after H/I. Mice were subjected to 15-min H/I and received 2 i.p. administrations of 0.375 g Tri-DHA/kg (n=6) vs. saline (n=5). At 8 wk. after H/I mice were sacrificed and brains were fixed with 4% paraformaldehyde and 10 μm-thick slices were cut and preserved. Nissl staining was used for identifying neuronal and brain structure. As described in Methods of Example 5 right brain tissue loss in relation to the contralateral hemisphere was calculated and expressed as a percentage. Each bar represents the mean±SEM. *p<0.05.

FIG. 17 are graphs that illustrate navigational memory assessment in vivo. Eight weeks after stroke, neuronal function is maintained and is much better in mice that had been treated initially with pure omega-3 Tri-DHA (0.375 mg/g Tri-DHA) after stroke. Results were much better with Tri-DHA has compared to Tri-EPA. Mice were given 0.375 mg/g Tri-DHA immediately after H/I injury and 1 hour later.

FIG. 18A-18C are bar graphs that illustrate the reduction of acute MI in vivo after administration of n-3 TG. FIG. 18A is a bar graph that illustrates the reduction of infarct size area (%) for (left) control and (right) after administration of n-3 TG (1.5 g/kg body weight) in the mouse heart after H/I injury. FIG. 18B is a bar graph that illustrates a decrease in LDH release which is a marker for heart cell damage for (left) control and (right) after administration of n-3 TG. FIG. 18C is a bar graph that illustrates n-3 TG maintenance of heart function via fractional shortening (%) for (left) control and (right) after administration of n-3 TG. Each bar represents the mean±SEM. *p<0.01.

FIG. 19 is a bar graph that illustrates increase in gene expression and mouse heart protein expression of BCL-2 which is an anti-apoptotic marker for (left) normoxia, (middle) control, and (right) after administration of 300 mg n3TG/100 ml post ischemic event. Each bar represents the mean±SEM. *p<0.05.

FIG. 20 is a diagram that illustrates an in vivo left anterior descending coronary artery (LAD) occlusion model and a time line for experimental design of reperfusion experiments after induced ex vivo ischemia in isolated hearts.

FIG. 21 is a graph showing that n-3 TG (0.75 mg/g body weight) decreases mitochondrial production of reactive oxidation species [ROS].

FIG. 22A-22C show the effects on hearts in vivo of acute n-3 TG emulsions administered after H/I in the in vivo left anterior descending coronary artery (LAD) occlusion model. Mice were subjected to LAD occlusion for 30 min followed by reperfusion period (48 h) in vivo with or without acute n-3 TG (1.5 g/kg body weight) emulsion injection. FIG. 22A is a bar graph that shows that hearts isolated at 48 h of reperfusion and subjected to TTC staining n=3 mice/group. *P<0.05. FIG. 22B is a bar graph that shows total plasma LDH levels at 48 hrs. n=3 mice/group. *P<0.05. FIG. 22C is a bar graph that quantifies fractional shortening percentage detected by echocardiogram just before sacrifice at 48 hrs. n=3 mice/group. *P<0.05. Data represent means±SD.

FIG. 23A-23B shows the effect of n-3 TG emulsion on an ex vivo cardiac ischemia/reperfusion model. FIG. 23A is a bar graph that shows myocardial ischemic injury by measuring left ventricular developed pressure (LVDP) recovery in hearts subjected to ischemia/reperfusion treated with or without n-3 TG (wherein n-3 TG=90% TG and 30% DHA)/300 mg/TG/100 ml) emulsion during reperfusion. *P<0.05. FIG. 23B is a bar graph that shows the level of LDH release during reperfusion time. *P<0.05. Four hearts per group were studied. Data represent means±SD.

FIG. 24A-24C illustrate signaling pathways. FIG. 24A is a photograph that shows western blot analysis of p-AKT and p-GSK3β in hearts subjected to ischemia/reperfusion injury with or without n-3 TG (wherein n-3 TG=90% TG and 30% DHA) (300 mg TG/100 ml) emulsion treatment. FIG. 24B is a bar graph that shows that p-AKT was increased by n-3 TG treatment. FIG. 24C is a bar graph that shows that GSK3b were increased by n-3 TG treatment. *P<0.05. Three hearts per group were studied. Data represent means±SD.

FIG. 25A-25C illustrate markers for apoptosis and autophagy. FIG. 25A is a photograph that shows western blot analysis of Bcl-2 and Beclin-1 in hearts subjected to I/R injury with or without n-3 TG (wherein n-3 TG=90% TG and 30% DHA) (300 mg TG/100 ml) emulsion administered during reperfusion time. FIG. 25B is a bar graph that shows increased Bcl-2 and FIG. 25C is a bar graph that shows decreased Beclin-1 protein expression in n-3 TG (300 mg TG/100 ml) treated hearts. *P<0.05. Three hearts per group were studied. Data represent means±SD.

FIG. 26A-26B are bar graphs that show release of the injury marker LDH. FIG. 26A is a bar graph that shows LDH release (fold change) compared to I/R control (reported in FIG. 26A-26B). p-AKT inhibitor (10 μM LY-294002) increased LDH. Treatment with GSK3β inhibitor (3 μM GSK2B1) significantly inhibited LDH release. P<0.05. Data represent means±SD. FIG. 26B is a bar graph that illustrates LDH release (fold change) compared to n-3 TG (300 mg TG/100 ml) I/R (reported in FIG. 26A-26B). p-AKT inhibitor abolished the beneficial effect of n-3 TG. GSK3β inhibitor (3 μM GSK2B1). Rosiglitazone treatment entirely reversed the protective effect of n-3 TG (300 mg TG/100 ml). P<0.05. Six hearts per group were studied. Data represent means±SD. *P<0.05 I/R vs treatments.

FIG. 27A-27C illustrate examples of transcription factors expression. FIG. 27A is a photograph that shows western blot analysis of PPAR-γ and HIF-1a in hearts subjected to I/R injury with or without n-3 TG (wherein n-3 TG=90% TG and 30% DHA) (300 mg TG/100 ml) emulsion administered during reperfusion time. FIG. 27B is a bar graph that shows HIF-1a. FIG. 27C is a bar graph that shows PPAR-γ expression which were decreased by n-3 TG (300 mg TG/100 ml) administration. *P<0.05. Three hearts per group were studied. Data represent means±SD.

Before the present embodiments of the invention are described, it is to be understood that the inventions are not limited to the particular processes, compositions, or methodologies described, as these may vary. The terminology used in the description is for the purpose of describing the particular versions or embodiments only, and is not intended to limit the scope of the present invention. Unless otherwise defined, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details.

### 1. DEFINITIONS

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Eric Kandel et al., eds., Principles of Neural Science, 4th ed., McGraw-Hill/Appleton & Lange: New York, N.Y. (2000).

As used herein, “omega-3 diglyceride oil” or “omega-3 triglyceride oil” means an omega-3 oil comprising di- or triglycerides, or combinations thereof, that have less than 10% omega-6 oil, preferably less than 5% omega-6 oil. As used herein, “omega-3 oils” means any omega-3 fatty acid, including free omega-3 fatty acids and omega-3 triglycerides, diglycerides and monoglycerides.

As used herein, “omega-3 lipid-based emulsion” is an oil-in-water emulsion comprising at least 7% to about 35% (and up to 100%) omega-3 oil by weight in grams per 100 ml of emulsion. Preferably the omega-3 lipid-based emulsion comprises at least 20% omega-3 oil, the omega-3 oil is a triglyceride and the emulsion comprises less than 10%, preferably less than 5% omega-6 fatty acids.

As used herein, “omega-3 oils” means any omega-3 fatty acid, including free omega-3 fatty acids and omega-3 triglycerides, diglycerides and monoglycerides.

As used herein, the term “omega-3 fatty acid” means a polyunsaturated fatty acid wherein one of the carbon-carbon double bonds is between the third and fourth carbon atoms from the distal end of the hydrocarbon side chain of the fatty acid. Examples of “omega-3 fatty acid” include α-linolenic acid (18:3n-3; α-ALA; Δ^{3,6,9}), eicosapentaenoic acid (20:5n-3; EPA; Δ^{5,8,11,14,17}), docosahexaenoic acid (22:6n-3; DHA) and docosapentaenoic acid (22:5n-3; DPA; Δ^{7,10,13,16,19}), wherein EPA and DHA are most preferred. Omega-3 fatty acids having at least 20 carbon atoms are herein called “long chain omega-3 fatty acids.”

As used herein, the term “omega-3 triglyceride” or “omega-3 diglyceride” or “omega-3 monoglyceride” refers to a triglyceride or diglyceride or monoglyceride, respectively, comprising at least one omega-3 fatty acid esterified with a glycerol moiety. As used herein, the term “omega-3 tri/diglyceride” means that omega-3 fatty acid comprises an omega-3 triglyceride and/or a diglyceride or any combination thereof.

As used herein, the amount of omega-3 oil or omega-6 oil in the lipid-based oil-in-water emulsion is expressed by weight in grams of omega-3 or omega-6 oil per 100 mL emulsion.

The amount of glyceride (mono-, di-, or triglyceride) in an omega-3 oil or omega-6 oil is expressed as the percentage of the glyceride by weight per total weight of the omega-3 or omega-6 oil.

The amount of fatty acid such as EPA or DHA in a glyceride (mono-, di-, or triglyceride) is expressed as the % of the total acyl groups of the respective glyceride. This measurement can also be given as wt.-%.

The abbreviations “n-3-TG” and “n-3TG90-DHA30” are used interchangeably in the examples and the figures herein; both refer to a particular omega-3 triglyceride emulsion of the present invention, that is, a 10% fish oil-in-water-based emulsion (10 g of omega-3 TG/100 mL) prepared for intravenous administration wherein >90% of the n-3 oil is triglyceride (TG) by weight in grams per total weight of the omega-3 oil and in which about 30% of the total acyl groups of the TG are DHA. N-3 TG has about 28% of the total acyl groups of the TG as EPA.

“Omega-3 triglyceride emulsions” of the present invention for use in the present methods means an oil-in-water emulsion comprising (a) at least 7% to about 35% (and up to 100%) omega-3 oil and less than 10% omega-6 oil by weight in grams per 100 ml of emulsion; (b) the omega-3 oil comprises at least 20% to 100% triglyceride by weight per total weight of the omega-3 oil, and at least 20% to 100% of the total acyl-groups of the omega-3 triglycerides consist of DHA or EPA; (c) the omega-3 oil comprises less than 10% omega-6 fatty acids; and (d) the mean diameter of lipid droplets in the emulsion is less than about 5 microns. In preferred embodiments the omega-3 triglyceride emulsions comprise less than 5% and preferably less than 3% omega-6 oil by weight in grams per 100 grams of emulsion. A specific omega-3 triglyceride emulsion herein described and tested in vivo and ex vivo is the “N-3 TG emulsions”. In other embodiments the omega-3 triglyceride emulsions of the present invention contain lipid particles having a diameter of about 100-400 nanometer (up to 1-5 microns), with an average size of 300 nanometers. For parenteral application, median lipid droplet sizes may be less than about 1 μm and preferably in the range from about 100-500 nm, more preferably from about 100 nm to about 400 nm, most preferably from about 200 nm to about 350 nm. For other applications, such as transdermal applications, mean diameter of the lipid droplet can be larger, for example, from about 1 μm and 5 μm.

As used herein, “hypoxia” refers to a shortage of oxygen in the body or in a specific organ or tissue.

As used herein, “ischemia” refers to insufficient blood flow to provide adequate oxygenation. The most common causes of ischemia are acute arterial thrombus formation, chronic narrowing (stenosis) of a supply artery that is often caused by atherosclerotic disease, and arterial vasospasm. As blood flow is reduced to an organ, oxygen extraction increases. When the tissue is unable to extract adequate oxygen, the partial pressure of oxygen within the tissue fails (hypoxia) leading to a reduction in mitochondrial respiration and oxidative metabolism. Further, in many acute situations of organ ischemia-hypoxia (e.g., stroke, myocardial infarction, intestinal volvulus, etc.) the patient is far too ill to have oral or enteral administration of therapeutic agents and thus needs parenteral injections, such as from lipid emulsions for immediate action.

As used herein, “hypoxia-ischemia” refers to the occurrence of both hypoxia and ischemia in a tissue or organ.

As used herein, “perfusion buffer” refers collectively to washout, preservation, intracellular and flush solutions devised and evaluated for cold storage of an isolated organ or tissue that has been removed from the body that is intended for transplantation. The isolated organ/tissue is perfused, reperfused, stored or otherwise contacted immediately after removal from the body with such a buffer or solution. The use of the term “intracellular” solutions is due to their resemblance, in some respects, to intracellular fluid. Experiments in which isolated hearts are subjected to hypoxia/ischemia and then reperfused are described in Example 8.

As used herein, “perfusion” refers to perfusing an isolated organ or tissue after isolation from the body, and “reperfusion” refers to subsequent perfusions after the organ or tissue has undergone an ischemic event.

As used herein, “n-3 glyceride perfusion emulsions” of the present invention refer to perfusion buffers that comprise less than 7% n-3 oil by weight per 100 ml of emulsion, and less than 10% n-6 oil by weight per 100 ml of emulsion.

As used herein, “tissue” is used herein to mean any controlled medical support product or biological substance such tissues, biological specimens or other medical products that require special conditions during transport.

As used herein, an “organ” is a collection of cells and tissues joined in a structural unit to serve a common function. Organs include any organ that can be transplanted including the heart, kidneys, liver, lungs, pancreas, intestine, and thymus. Tissues include bones, tendons (both referred to as musculoskeletal grafts), composite tissue allografts, cornea, skin, heart valves, nerves and veins. A “transplant organ” as used herein may refer to an organ that is also a donor organ and/or an organ that is intended for transplantation. “Transplant organ” may refer to a donor organ that has yet to be transferred to a recipient.

As used herein, “reperfusion damage” or “reperfusion injury” are used interchangeably herein to refer to damage caused with restoration of blood supply to hypoxic-ischemic (H/I) tissues either in vivo after an ischemic event or ex vivo in isolated organs and tissues. An ischemia-reperfusion injury can be caused, for example, by a natural event (e.g., restoration of blood flow following a myocardial infarction), a trauma, or by one or more surgical procedures or other therapeutic interventions that restore blood flow to a tissue or organ that has been subjected to a diminished supply of blood. Such surgical procedures include, for example, coronary artery bypass graft surgery, coronary angioplasty, organ transplant surgery and the like.

### 2. BACKGROUND

While not wishing it to be bound by theory, it is believed that organ death or injury is frequently precipitated by hypoxia-ischemia, or associated with reperfusion damage, cardiac infarct, organ transplantation, endothelial dysfunction, impaired organ micro perfusion, increased risk of thrombus formation, or ectopic fat deposition, etc. Ectopic fat depositions usually occur in organs not specialized in fat deposition, such as liver, pancreas, or heart.

Typically, post-operative and post-traumatic conditions as well as severe septic episodes are characterized by a substantial stimulation of the immune system ischemia reperfusion syndrome, and tendency for thrombosis formation. The immune response is activated by the release of pro-inflammatory cytokines (e.g., tumor necrosis factor and interleukins) which at high levels may cause severe tissue damage.

In such clinical conditions, it is of particular importance to provide exogenous lipids that are hydrolyzed and eliminated faster than endogenous lipids (to avoid excessive increases of plasma triglyceride concentration). These lipids supply omega-3 fatty acids capable of reducing cytokine production as well as cytokine toxicity on tissues. Thus, fatty acids are optimally administered via lipid glycerides such as triglycerides. The fatty acids are released and used after the lipids are catabolized in the body via lipolysis. This effect is obtained when fatty acids are cleaved from the lipid molecules and incorporated (in free form or as components of phospholipids) in cell membranes where they influence membrane structure and cell function, serve as secondary messengers (thus affecting regulation of cell metabolism), influence the regulation of nuclear transcription factors, and are precursors of eicosanoids. Thus, it is desirable that this process takes place as quickly as possible.

The human body is capable of synthesizing certain types of fatty acids. However, long chain omega-3 and omega-6 are designated as “essential” fatty acids because they cannot be produced by the human body and must be obtained through other sources. For example, fish oils from cold-water fish have high omega-3 polyunsaturated fatty acids content with lower omega-6 fatty acid content. Table 1, supplied by Fresenius Kabi, describes the makeup of the n-3 TG and n-6 TG used in the experiments described in Example 6 and Example 8. To date no fish oil has been reported that has over 10% omega-6 fatty acids. The di- and tri-glyceride omega-3 emulsions of the present invention, when made from fish oil, use fish oil with 10% or less omega-6 fatty acid. Most vegetable oils (i.e., soybean and safflower) have high omega-6 polyunsaturated fatty acids (most in the form of 18:2 (Δ^{9, 12})-linoleic acid) content but low omega-3 (predominantly 18:3 (Δ^{9, 12, 15})-α-linolenic acid) content.

Essential fatty acids may be obtained through diet or other enteral or parenteral administration. However, the rate of EPA and DHA omega-3 fatty acid enrichment following oral supplementation varies substantially between different tissues and is particularly low in some regions of the brain and in the retina especially when given as the essential fatty acid precursor, α-linolenic acid. Further, human consumption of omega-3 fatty acids has decreased over the past thirty years, while consumption of omega-6 fatty acids has increased, especially in Western populations.

Cao et al. (2005) “Chronic administration of ethyl docosahexaenoate decreases mortality and cerebral edema in ischemic gerbils,” Life Sci. 78(1):74-81 alleges that dietary docosahexaenoic acid (DHA) intake can decrease the level of membrane arachidonic acid (AA), which is liberated during cerebral ischemia and implicated in the pathogenesis of brain damage. Cao investigated the effects of chronic ethyl docosahexaenoate (E-DHA) administration on mortality and cerebral edema induced by transient forebrain ischemia in gerbils.

GB 2388026, incorporated herein by reference in its entirety, refers to use n-3 polyunsaturated fatty acids EPA and/or DHA in the preparation of an oral medicament for preventing cerebral damage in patients having symptoms of atherosclerosis of arteries supplying the brain.

Strokin, M. (2006), Neuroscience 140(2):547-53, incorporated herein by reference in its entirety, investigated the role of docosahexaenoic acid (22:6n-3) in brain phospholipids for neuronal survival.

WO 2004/028470 (PCT/US2003/030484), incorporated herein by reference in its entirety, purports to disclose methods and compositions which impede the development and progression of diseases associated with subclinical inflammation.

The following U.S. Patent applications are hereby incorporated by reference as if fully set forth herein: Provisional Application No. 60/735,862, filed Nov. 14, 2005; Provisional Application No. 60/799,677, filed May 12, 2006; application Ser. No. 11/558,568, filed Nov. 10, 2006; application Ser. No. 13/336,290, filed Dec. 23, 2011; Provisional Application No. 60/845,518, filed Sep. 19, 2006; PCT/US07/20364, filed Sep. 19, 2007; application Ser. No. 12/441,795, filed Dec. 8, 2009, now U.S. Pat. No. 8,410,181; application Ser. No. 13/783,779, filed Mar. 4, 2013; application Ser. No. 13/953,718, filed Jul. 29, 2013; application Ser. No. 14/102,146, filed Dec. 10, 2013; Provisional Application No. 61/767,248, filed Feb. 20, 2013; and PCT/US 14/17523, filed Feb. 20, 2014.

See also Qi, K. (2002) “Omega-3 triglycerides modify blood clearance and tissue targeting pathways of lipid emulsions,” Biochemistry 41: 3119-3127, incorporated herein by reference in its entirety, refers to omega-3 rich triglycerides which are recognized as having modulating roles in many physiological and pathological conditions.

It is now well-established that cerebral hypoxia-ischemia of sufficient duration to deplete high energy reserves in neural cells initiates a cascade of events over the hours to days of reperfusion that culminates in extensive death, both necrotic and apoptotic. These events include the generation of reactive oxygen species and oxidative damage to cells, release of inflammatory mediators and initiation of prolonged inflammatory reactions, and ongoing apoptosis that can continue for weeks to months. This applies to ischemic injury to organs in young, adult and elderly humans.

As an example, neuronal loss following hypoxia/ischemia is believed to result, at least in part, from elevated glutamate release and excitoxicity. Excess glutamate activation of N-methyl-D-aspartic acid (NMDA) receptors induces pro-apoptotic pathways and inhibits anti-apoptotic signaling pathways. Omega-3 fatty acids can modify a number of signaling pathways to effect transcriptional regulation. Not being bound by theory, since prior studies by the present inventors have shown that whole brain fatty acid profiles are not modified following acute administration of omega-3 triglyceride emulsions, it is believed that the omega-3 fatty acids protect neurons by modulating signaling pathways that counter the effects of hyper stimulated NMDA receptors, protection against free radical generation and consequent oxidative damage, maintaining mitochondrial function and thereby prevent/reduce post-ischemic inflammation and release of inflammatory mediators.

Recent evidence (see Qi K., Seo T., Al-Haideri M., Worgall T. S., Vogel T., et al. (2002) “Omega-3 triglycerides modify blood clearance and tissue targeting pathways of lipid emulsions,” Biochemistry 41: 3119-3127) also indicates that routes for blood clearance and tissue uptake of fish oil omega triglycerides are very different from those of omega-6 soy oil long chain triaglycerols (LCT). For example, removal of omega-3 very long chain triaglycerol (VLCT) emulsions from blood seems to depend far less on intravascular lipolysis than does LCT emulsions. While substantial amounts of both emulsions are delivered to tissues as intact triglyceride, this pathway is likely more important for omega-3 triglyceride particles. Omega-3 triglyceride particles, VLCT, are less dependent on “classical” lipoprotein receptor related clearance pathways, than are LCT. Fatty acid derived from omega-3 triglyceride appear to act as stronger inhibitors than LCT in sterol regulatory element (SRE) dependent gene expression-genes that are involved in both triglyceride and cholesterol synthesis.

### 3. SUMMARY OF EMBODIMENTS

The present invention provides certain methods of limiting or preventing cell death and cell/tissue damage resulting from hypoxic-ischemia by administering an omega-3 emulsion a (such as n-3 TG) to a subject in need of such treatment. “Limiting” as used herein includes decreasing and/or preventing. Among the effects of treating the ischemia are a reduction in cell death, decreased inflammation, reduction in infarct size, reduction in production of inflammatory cytokines, reduction in production of reactive oxygen species, and maintenance of mitochondrial integrity. The methods of present invention comprise administering an omega-3 triglyceride emulsion of the present invention after a hypoxic-ischemia insult. The present invention also provides, in those cases where the hypoxic-ischemic insult can be predicted, methods of limiting or preventing cell death and cell/tissue damage comprising administering an omega-3 triglyceride emulsion of the present invention before the hypoxic-ischemia insult.

When the hypoxic-ischemic insult is cerebral, the present invention limits neural cell death and/or limits neurological damage. Since the basic mechanisms of cell death following ischemia after an hypoxic-ischemic insult are similar in most bodily organs, the present invention also provides limiting cell death in other organs such as the heart, large and small intestines, kidney and lung following an hypoxic-ischemia insult. For example, after a colonic ischemic event due to acute mesenteric artery ischemia, chronic mesenteric artery ischemia or ischemia due to mesenteric venous thrombosis, the present invention provides a method of limiting intestinal cell death. Similar prevention of cell death would apply to myocardial infarction.

Prior studies have shown that omega-6 fatty acids such as omega-6 linoleic acids are far less effective in neuroprotection and cardiac protection when provided before and/or after an ischemic event than are omega-3 fatty acids These studies involved the administration of Intralipid®, a soy oil based emulsion containing 55% of its fatty acids as omega-6 linoleic acid, with a very low content of EPA and DHA (˜2%). Further, direct injection of free fatty acids (not in an emulsion), as compared to triglycerides or diglycerides, can have serious side effects, such as encephalopathy. By contrast, the omega-3 triglyceride emulsions of the present invention to be administered therapeutically to treat reperfusion injury following an ischemic event have less than 10% n-6 fatty acids (of any type), preferably less than 5%.

The examples herein show that administering omega-3 triglyceride emulsions, including n-3 TG (wherein >45% of total TG fatty acids are EPA+DHA) and Tri-DHA emulsions (wherein >98% of total TG fatty acids are DHA), after hypoxia/ischemia in vivo or ex vivo limited or prevented cell death and cell/tissue damage in the brain and heart; thus certain embodiments are directed to such methods. Omega-3 triglyceride emulsions for use in the present methods comprise (a) at least 7% to about 35% omega-3 oil and less than 10% omega-6 oil by weight in grams per 100 ml of emulsion; (b) the omega-3 oil comprises at least 20% to 100% triglyceride by weight per total weight of the omega-3 oil, and at least 20% wt.-% to 100% of the acyl-groups of the omega-3 triglycerides consist of DHA and/or EPA; (c) the omega-3 oil comprises less than 10% omega-6 fatty acids; and (d) the mean diameter of lipid droplets in the emulsion is less than about 5 microns. All of the enumerated omega-3 emulsions come within the scope of the invention.

Omega-3 Triglyceride Emulsions for Treating Reperfusion Injury after Hypoxia/Ischemia (H/I)

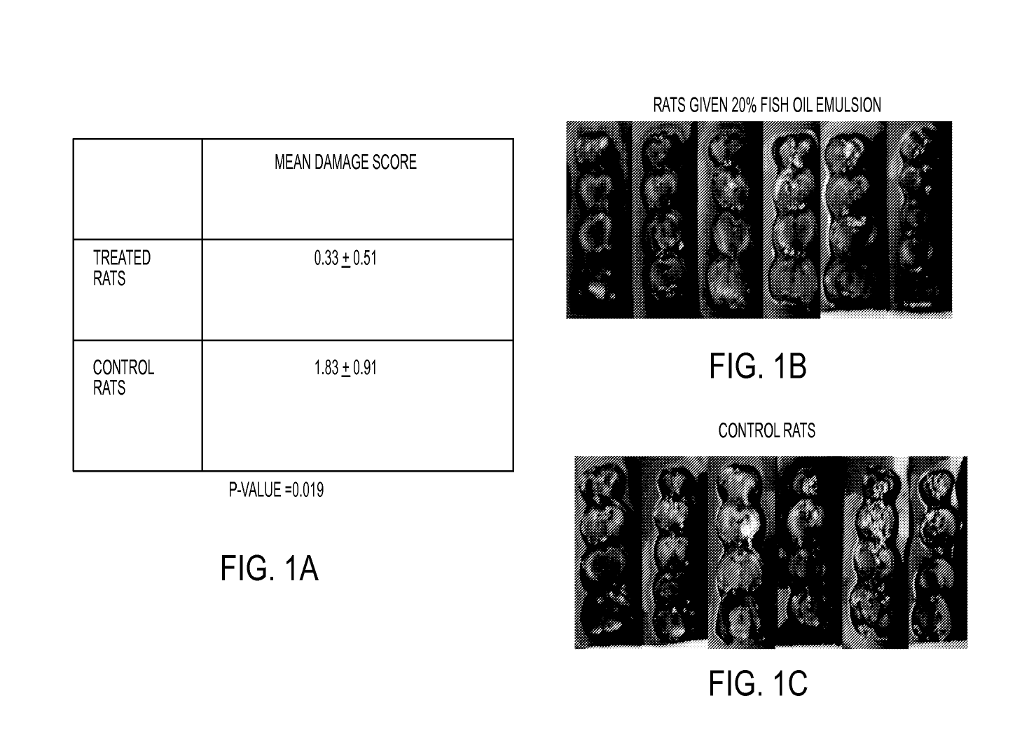

In Examples 1-4, Wistar rats of both genders were subjected to unilateral (right) carotid artery ligation and cerebral H/I was induced. Immediately after ligation, six rats were given 50 mg 20% omega-3 triglyceride lipid-based emulsion (0.25 cc), a 20% long chain omega-3 triglyceride-based formula having >45% of total omega-3 fatty acid as eicosapentaenoic acid (EPA) and docosahexaenoic acid (DHA). The results of these experiments are shown in FIGS. 1-4. Additional experiments were conducted examining the treatment of cerebral H/I with omega-3 triglyceride emulsions. In experiments described in Example 6, omega-3 fish oil-based and n-6 soy oil-based emulsions were compared. The n-6 TG emulsions were produced from soybean oil rich in n-6 FA: linoleic acid constituting about 55% of total FA. In contrast, the n-3 TG fish oil emulsions comprised 10% omega-3 triglyceride oil by weight in grams per 100 ml emulsion, wherein the omega-3 triglyceride oil comprises at least 90% triglyceride by weight per total weight of the omega-3 oil, and are rich in EPA (up to 28%) and DHA (up to 30%) (measured as % of the total acyl groups on the triglyceride). It was discovered that the n-3 TG emulsions had a neuroprotective effect against cerebral infarction and reperfusion damage following cerebral H/I, while the n-6 TG emulsion did not. Further, experiments in Example 7 show that a pure triglyceride DHA emulsion (Tri-DHA) had a therapeutic neuroprotective effect while pure triglyceride EPA (Tri-EPA) did not. Some of these results are described in Williams (see Williams J J, Mayurasakorn K, Vannucci S J, Mastropietro C, Bazan N G, et al. (2013) N-3 Fatty Acid Rich Triglyceride Emulsions Are Neuroprotective after Cerebral Hypoxic-Ischemic Injury in Neonatal Mice, PLoS ONE 8(2): e56233. doi:10.1371/journal.pone.0056233). In Example 8, experiments are described showing that administration of n-3 TG (TG90/DHA30) in vivo after hypoxic-ischemic injury decreased infarct size and LDH release which is a marker for heart cell damage, and also maintained heart function at more normal levels. Ex vivo reperfusion experiments were also conducted showing that reperfusion with n-3 TG (with ˜48% of TG fatty acids being EPA+DHA) and in perfusion buffer) also had a protective effect Based on these discoveries certain other embodiments are directed to new omega-3 Triglyceride-DHA-based oil-in-water emulsions (Tri-DHA emulsions) and to their therapeutic use in treating H/I and reperfusion injury, including in organ transplants.

Based on these observations showing effective treatment of both cerebral and cardiac hypoxia-ischemia and reperfusion damage, certain embodiments are directed to omega-3 triglyceride emulsions (including n-3TG and Tri-DHA lipid-based oil-in-water emulsions) wherein: (a) the emulsion comprises at least 7% to about 35% omega-3 oil and less than 10% omega-6 oil by weight in grams per 100 ml of emulsion; (b) the omega-3 oil comprises at least 20% to 100% triglyceride by weight per total weight of the omega-3 oil, and at least 20% wt.-% to 100% of the acyl-groups of the omega-3 triglycerides consist of DHA; (c) the omega-3 oil comprises less than 10% omega-6 fatty acids; and (d) the mean diameter of lipid droplets in the emulsion is less than about 5 microns.

In certain embodiments the omega-3 oil component comprises 7%, 10%, 20%, 30% or 35% (up to 100%) of the emulsion by weight in grams per 100 ml of emulsion; the triglyceride component in the described omega-3 triglyceride emulsions of the present invention is 20-50%, 50-75%, 75-90%, 90-95%, 95%-100% weight per total weight of the omega-3; and the DHA content of the omega-3 triglyceride is from 20-50%, 50-75%, 75-90%, 90-95%, 95%-100% of the total acyl groups of the TG (also measured as wt. %). It is important to note that in these omega-3 triglyceride emulsions it is not necessary to exclude EPA in order to treat hypoxia/ischemia. However, in the case of triglycerides with pure DHA, EPA would be excluded. For the omega-3 triglyceride emulsions the presence of at least 20% triglycerides in the omega-3 oil of which at least 20% are DHA is needed to treat hypoxia/ischemia and reperfusion damage.

Yet other embodiments include methods of treating H/I and reperfusion damage including any cerebral or cardiac H/I including stroke and myocardial infarction, respectively; and also methods of treating H/I in organs or tissue before it is harvested for transplantation, or after transplantation to minimize cell death and cell damage. In some embodiments the methods include (a) identifying a subject who has undergone hypoxia-ischemia, (b) administering to the subject a pharmaceutical composition comprising a therapeutically effective amount the described omega-3 triglyceride emulsions of the present invention to reduce reperfusion damage caused by the hypoxia-ischemia. In some embodiments the hypoxia-ischemia causes cerebral hypoxia-ischemia including stroke and the described omega-3 triglyceride emulsion is administered as soon as possible after the hypoxia-ischemia, such as within 20 minutes, or less than 1 hour, less than 2 hours, less than 3 hours and less than 4 hours after the H/I, and in some cases less than 6 hours after. In other embodiments the hypoxia-ischemia is in the heart and it causes myocardial infarction. Again, treatment is optimal as soon as possible after the diagnosis of the cardiac hypoxia-ischemia to reduce reperfusion damage. In some embodiments the described omega-3 triglyceride emulsions are administered to reduce cell damage or cell death either before tissue or organs are harvested for transplantation following organ transplant or hypoxia-ischemia in organs including organ selected from the group consisting of brain, heart, kidney, spinal cord, large or small intestine, pancreas and lung.

In some embodiments the therapeutically effective amount is from about 0.05 g/kg/administration to about 4 g/kg/administration, but higher doses can be administered if a crisis warrants treatment as the emulsions are non-toxic. In some cases the omega-3 emulsions of the present invention including n-3 DG and n-3 TG emulsions can be administered continuously for a period of time after the H/I.

Other embodiments include a method comprising: (a) identifying a subject who is at risk of having a cerebral hypoxia-ischemia, and (b) administering to the subject a pharmaceutical composition comprising a therapeutically effective amount of the described omega-3 triglyceride emulsion, thereby reducing the risk of the subject developing the reperfusion damage caused by the hypoxia-ischemia. Subjects at risk of developing cerebral H/I and cardiac H/I can often be identified before they develop H/I; such subjects come within treatment with the present methods. Another method comprises (a) identifying a subject who has inflammation in an organ, and (b) administering to the subject a pharmaceutical composition comprising a therapeutically effective amount of the described omega-3 triglyceride emulsion, thereby reducing the inflammation in the subject. Yet another method comprises administering to a subject an amount of a pharmaceutical composition comprising the described omega-3 triglyceride emulsion, to reduce production of reactive oxygen species or inflammatory cytokines in the blood or an organ in the subject.

Sources of omega-3 fatty acids may be from any suitable source such as from fish oils or other natural oils or they may be synthesized. The omega-3 triglyceride oils in the present emulsions have less than 10%, preferably less than 5% omega-6 oil, and the emulsions themselves similarly have less than 10%, preferably less than 5% omega-6 oil. Although EPA and DHA are preferred omega-3 fatty acids, other omega-3 fatty acids may be used. Suitable exemplary fish oils include oils from cold-water fish such as salmon, sardine, mackerel, herring, anchovy, smelt and swordfish. Fish oils generally contain glycerides of fatty acids with chain lengths of 12 to 22 carbons. Highly purified fish oil concentrates obtained, for example, from sardine, salmon, herring and/or mackerel oils may have an eicosapentaenoic acid (EPA) content of from about 20 to 40 wt.-%, preferably at least 25 wt.-%, and a docosahexaenoic acid (DHA) content of >10%, preferably at least 12%, based on the fatty acid methyl esters of the fish oil concentrate as determined by gas chromatography (percent by area). U.S. Pat. No. 6,159,523, incorporated herein by reference in its entirety, discloses a method for making fish oil concentrates. Generally, the amount of the polyunsaturated fatty acids of the omega-6 series (such as linoleic acid) in natural fish oils is low, i.e. less than 10%, preferably less than 5%. In the described Tri-DHA emulsions used in the present embodiments, the amount of omega-6 oil in the emulsions is less than 10%, preferably less than 5%. The amount of omega-6 oil in the fish oil is also less than 10%. To make omega-3 triglyceride with a high % DHA of more than about 25% of the total acyl groups of the TG, the oil is typically synthesized or obtained from selectively bred algae.

Methods of the present invention preferably include administering the omega-3 triglyceride emulsions including the particularly described Tri-DHA emulsions of the present invention by any suitable route including enterally (for example, orogastric or nasogastric) or parenterally (for example, subcutaneous, intravenous, intramuscular, intraperitoneal). Most preferably the emulsion is administered intravenously.

Omega-3 triglyceride emulsions of the present invention are preferably provided at a dose capable of providing a protective benefit. Those skilled in the art would be able to determine the appropriate dose based on the experimental data presented herein. However, for example a suitable effective and tolerable dose for a human would be about 0.05 g/kg to about 4.0 g/kg. Higher doses may be given as necessary. Administration may be continuous or in the form of one or several doses per day. One skilled in the art would appreciate appropriate dosage and routes of administration based upon the particular subject and condition to be treated.

Omega-3 triglyceride emulsions of the present invention are preferably administered parenterally and/or enterally as soon after the ischemic insult as possible (or in some embodiments, before the insult when it can be predicted). The emulsion may be administered to prevent/reduce tissue damage and cell death from reperfusion injury after hypoxia-ischemia, including from organ transplant, in any organ including brain, heart, kidney, spinal cord, large or small intestine, pancreas and lung. For example, in a preferred embodiment an omega-3 triglyceride emulsion is administered from 0-20 minutes to two hours after the insult. In some embodiments the emulsion is administered within 1 hour, 2-3 hours, and 3-4 hours and in less than six hours after the insult. The present invention also provides for multiple administrations of the omega-3 triglyceride emulsion on the same day or even continuous infusion since these emulsions are not toxic. Routine experimentation will determine the optimal dose for the injury. For example, the emulsion may be first administered within 20 minutes of the insult, followed by a second administration 1-24 hours after the insult. Cerebral H/I administration of the described omega-3 triglyceride emulsions is optimal within 4 hours.

In another embodiment of the invention, methods of limiting or preventing cell death and cell/tissue damage resulting from hypoxic-ischemia further comprise administering an omega-3 triglyceride emulsion of the present invention including the described Tri-DHA emulsion, in conjunction with standard available therapies (such as surgery and angioplasty) and/or medications given to prevent or treat hypoxia-ischemia. For example, the following drugs are often administered to prevent or treat strokes: antiplatelet medications such as aspirin, clopidogrel, dipyridamole, ticlopidine; anticoagulants such as heparin and warfarin; and thrombolytic agents such as tissue plasminogen activator (tPA).

Preparations of omega-3 triglyceride emulsions suitable for intravenous delivery are known in the art. Omega-3 lipid-based emulsions according to the invention may be oil-in-water (o/w) emulsions in which the outer continuous phase consists of distilled water purified or sterilized for parenteral purposes. Such oil-in-water emulsions may be obtained by standard methods, i.e. by mixing the oil components followed by emulsification and sterilization. The pH value of the lipid emulsion may be adjusted to a physiologically acceptable value, preferably to a pH of from about 6.0 to about 9.0, more preferably from about 6.5 to about 8.5. Auxiliary agents and additives may be added to the oil mixture prior to emulsification or prior to sterilization. The omega-3 emulsions are preferably isotonic. Methods for making emulsions are well known in the art and are described for example in Kirk-Othmer, Encyclopedia of Chemical Technology, 3^{rd} Ed., v. 8, pp. 900-933 (1979). See also U.S. Pat. Nos. 2,977,283; 3,169,094; 4,101,673; 4,563,354; 4,784,845; 4,816,247, all of which are incorporated by reference in their entirety.

Omega-3 lipid-based emulsions according to the invention can be prepared by known standard procedures with inertization. Typically, first the lipids, emulsifier and other auxiliary agents and additives are mixed and then filled up with water with dispersing. The water may optionally contain additional water-soluble components (e.g. glycerol). The omega-3 emulsions of the present invention have lipid particles less than 5 microns in diameter. Preferably the omega-3 triglyceride emulsions of the present invention contain lipid particles having a diameter of about 100-400 nanometer, with an average size of 300 nanometers. For parenteral application, median lipid droplet sizes may be less than about 1 μm and preferably in the range from about 100-500 nm, more preferably from about 100 nm to about 400 nm, most preferably from about 200 nm to about 350 nm. For other applications, such as transdermal applications, mean diameter of the lipid droplet can be larger, for example, from about 1 μm and 5 μm.

The present invention also provides omega-3 triglyceride emulsions suitable for enteral or parenteral administration to provide a protective benefit on cells against cell death following a hypoxic-ischemic insult. Omega-3 triglyceride emulsions of the present invention comprise at least 7%, 10%, 20% (up to 100%) by weight of omega-3 oil, preferably 7% to about 35% omega-3 oil by weight in grams per 100 ml of emulsion. In some embodiments the omega-3 oil comprises at least 10%, preferably at least 20% (up to 100%) omega-3 tri/diglyceride. Fatty acids in the omega-3 tri/diglyceride preferably comprise at least 20%-40% (up to 100%) EPA and/or DHA. In certain embodiments the triglyceride has at least 20% DHA 30%, 40%, 50%, 75% and up to 99% DHA. Preferably, omega-3 emulsions of the present invention are sterile and have a particle size that is less than 5 microns, preferably between 100-400 nanometer mean diameter, with an average size of 300 nm.

### Pharmaceutical Formulations

The term “the omega-3 lipid-based emulsions” generally include the specifically described “n-3 TG” and DG emulsions, including the “Tri-DHA” emulsions. Administration of the omega-3 triglyceride- and DG-based emulsions of the present invention, including the described Tri-DHA emulsions, may be either enteral, parenteral, or transdermal. Any method known in the art could be used including local administration or perfusion of an organ in vivo. The methods of administration of a pharmaceutical composition of the omega-3 lipid-base emulsions may further comprise any additional administrations of other conventional stroke treatment or preventative medication.

The omega-3 lipid-based emulsions of the present may contain from about 2 wt.-% to about 5 wt.-% of a stabilizing or isotonizing additive, such as a polyhydric alcohol, based on the emulsion. Preferred stabilizing or isotonizing additives include glycerol, sorbitol, xylitol or glucose. Glycerol is most preferred.

In addition to distilled water, the omega-3 lipid-base emulsions of the present invention may contain conventional auxiliary agents and/or additives, such as emulsifiers, emulsifying aids (co-emulsifiers), stabilizers, antioxidants, and isotonizing additives.

Emulsifiers may include physiologically acceptable emulsifiers (surfactants) such as phospholipids of animal or vegetable origin. Examples of phospholipids are egg yolk lecithin, a biologic phospholipid, a phosphatidylcholine with fixed fatty acyl chain composition, a glycophospholipid or a phosphatidylethanolamine. Particularly preferred are purified lecithins, especially soybean lecithin, egg lecithin, or fractions thereof, or the corresponding phosphatides. The emulsifier content may vary from about 0.02 wt.-% to about 2.5 wt.-%, preferably from about 0.6 wt.-% to about 1.5 wt.-% and most preferably about 1.2 wt.-%, based on the total emulsion. In one embodiment the emulsifier is 1.2 mg of egg yolk lecithin/100 ml emulsion.

Alkali metal salts, preferably sodium salts, of long chain, C₁₆ to C₂₈ fatty acids may also be used as emulsifying aids (co-emulsifiers). The co-emulsifiers are employed in concentrations of from about 0.005 wt.-% to about 0.1 wt.-%, preferably about 0.02 wt.-% to about 0.04 wt.-%, based on the total emulsion. Further, cholesterol or a cholesterol ester alone or in combination with other co-emulsifiers may be employed as an emulsifying aid in a concentration of from about 0.005 wt.-% to about 0.1 wt.-%, preferably from about 0.02 wt.-% to about 0.04 wt.-%, based on the emulsion.

The omega-3 lipid-based emulsions of the present invention may further comprise an effective amount of an antioxidant, such as vitamin E, in particular α-tocopherol (the most active isomer of vitamin E in humans) as well as β- and γ-tocopherol, and/or ascorbyl palmitate as antioxidants and thus for protection from peroxide formation. The total amount of alpha tocopherol may be up to 5000 mg per liter. In a preferred embodiment the total amount of said antioxidant is from about 10 mg to about 2000 mg, more preferably from about 25 mg to about 1000 mg, most preferably from about 100 mg to 500 mg, based on 100 g of lipid.

Since omega-3 emulsions contain low concentrations of alphatocopherol as an anti-oxidant agent, in separate experiments an equivalent dose of pure alpha-tocopherol to match the content of n-3 emulsion content (0.8 g/L) was given to neonatal mice by i.p. injection of alpha-tocopherol (Vital EH, Intervet, Schering Plough) at a dose of 5 mg alpha-tocopherol/kg body weight, the amount contained in each i.p. injection of the n-3 TG emulsions. TTC staining was used to compare the extent of cerebral H/I injury in alpha-tocopherol-treated and saline-treated neonatal mice. There was no significant difference in infarct volume between brains in alpha-tocopherol injected mice compared to saline treated mice (data not shown).

The omega-3 triglyceride emulsions of the invention may be administered orally, enterally, parenterally, transdermally, intravascularly, intravenously, intramuscularly, intraperitoneally or transmucosally, and are preferably administered by intravenous injection. Thus the present invention also relates to a pharmaceutical composition comprising omega-3 diglyceride emulsions as described herein, preferably for injection into the human or animal body.

Pharmaceutical compositions of the invention may further comprise various pharmaceutically active ingredients. In particular, the pharmaceutically active ingredient may be delivered to a particular tissue of the body (drug targeting) in combination with emulsions of the present invention. The omega-3 lipid-base emulsions may include carriers for such targeted tissue treatment. Suitable carriers may be, for example, macromolecules linked to the emulsion droplet, lipid microspheres comprising soybean oil or lecithin or fish oil U.S. Pub. No. 2002/0155161, incorporated herein by reference in its entirety, discloses tissue-targeted delivery of emulsions.

The pharmaceutical composition may be formulated into a solid or a liquid dosage form. Solid dosage forms include, but are not limited to, tablets, pills, powders, granules, capsules, suppositories, and the like. Liquid dosage forms include, but are not limited to liquids, suspensions, emulsions, injection preparations (solutions and suspensions), and the like. The choice of dosage form may depend, for example, on the age, sex, and symptoms of the patient.

The pharmaceutical composition may optionally contain other forms of omega-3 Tri-DHA or diglyceride emulsions and/or additional active ingredients. The amount of omega-3 Tri-DHA/diglyceride emulsions or other active ingredient present in the pharmaceutical composition should be sufficient to treat, ameliorate, or reduce the target condition.

The pharmaceutically acceptable excipient may be any excipient commonly known to one of skill in the art to be suitable for use in pharmaceutical compositions. Pharmaceutically acceptable excipients include, but are not limited to, diluents, carriers, fillers, bulking agents, binders, disintegrants, disintegration inhibitors, absorption accelerators, wetting agents, lubricants, glidants, surface active agents, flavoring agents, and the like.

Carriers for use in the pharmaceutical compositions may include, but are not limited to, lactose, white sugar, sodium chloride, glucose, urea, starch, calcium carbonate, kaolin, crystalline cellulose, or silicic acid.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

In liquid pharmaceutical compositions of the present invention, the omega-3 emulsions of the present invention and any other solid ingredients are dissolved or suspended in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium gluconate, sodium lactate, sodium citrate or sodium acetate.

Selection of excipients and the amounts to use can be readily determined by an experienced formulation scientist in view of standard procedures and reference works known in the art.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations.

Pharmaceutical compositions of the invention may further comprise various pharmaceutically active ingredients. In particular, the pharmaceutically active ingredient may be delivered to a particular tissue of the body (drug targeting) in combination with micro emulsions of the present invention. Omega-3 emulsions may include carriers for such targeted tissue treatment. Suitable carriers may be, for example, macromolecules linked to the emulsion droplet, lipid microspheres comprising soybean oil or lecithin or fish oil. U.S. Pub. No. 2002/0155161, incorporated herein by reference in its entirety, discloses tissue-targeted delivery of emulsions.

Omega-3 TG and DG emulsions of the invention allow for rapid and efficient uptake of omega-3 fatty acids, including EPA and DHA, into cell membranes of organs and tissues. Accordingly, there is provided a method for delivering an emulsion of omega-3 Tri-DHA or diglycerides enriched with EPA or DHA to cells and organs by administering omega-3 emulsions of the present invention.

Lipolysis of emulsions of the invention facilitates the release of free omega-3 fatty acids and monoglycerides into the bloodstream or in cells. Free fatty acids may be transported into mitochondria for use as an energy source, or may be incorporated into cell membranes. Enriching cell membranes and phospholipids with omega-3 long chain polyunsaturated fatty acids (PUPA) may help promote or restore an adequate balance between omega-3 and omega-6 fatty acids. Incorporation of EPA and DHA also increases membrane fluidity and flexibility.

### 4. EXAMPLES

### Example 1

### 60 Minutes of Hypoxia-Ischemia

Postnatal day 19-21 Wistar rats of both genders were subjected to unilateral (right) carotid artery ligation. See Rice, J. E., 3rd, R. C. Vannucci, et al. (1981), “The influence of immaturity on hypoxic-ischemic brain damage in the rat,” Ann Neurol 9(2): 131-41 and Vannucci, S. J., L. B. Seaman, et al. (1996), “Effects of hypoxia-ischemia on GLUT1 and GLUT3 glucose transporters in immature rat brain,” Journal of Cerebral Blood Flow & Metabolism 16(1): 77-81.

Immediately after ligation, six rats were given 50 mg 20% omega-3 triglyceride emulsion (0.25 cc)(a 20% long chain omega-3 triglyceride-based formula having >45% of total omega-3 fatty acid as eicosapentaenoic acid (EPA) and docosahexaenoic acid (DHA)) via orogastric feeding tube, and six control rats were given 0.25 cc water, both enterally. The 20% omega-3 triglyceride emulsion was made placing 20 gm of omega-3 triglyceride in 100 ml of water, and emulsifying with 1.2 gm of egg yolk lecithin. Rats were allowed to recover for 2 hours and then they underwent hypoxia-ischemia for 60 minutes of 8% oxygen at a constant temperature. The six pre-treated rats were given another dose of 50 mg omega-3 triglyceride emulsion immediately after the hypoxia-ischemia and control rats were given 0.25 cc water. All rats were euthanized at 72 hours of reperfusion. The brains were removed and cut into 2 mm sections and stained with 2, 3, 5, Triphenyl-2H-tetrazolium chloride (TTC). TTC is a vital die that stains cells red that have respiring mitochondria. Dead tissue (infarct) appears white. The sections were scored as follows:

0—no evidence of edema or cell death

1—edema without cell death

2—edema with minimal cell death

3—edema with significant cell death.

All rats survived 60 minutes of hypoxia-ischemia. Six of the six control rats had edema and/or cell death with a mean score of 2+/−0.83 (standard deviation), while two of the six treated rats had damage with a mean score of 0.42+/−0.62 (p<0.005). FIG. 1.

### Example 2

### 65 Minutes of Hypoxia-Ischemia

Postnatal day 19-21 Wistar rats of both genders were subjected to unilateral (right) carotid artery ligation. Immediately after ligation, six rats were given 50 mg 20% omega-3 triglyceride emulsion (0.25 cc)(20% omega-3 fatty acid based formula having ≧40% of total omega-3 fatty acid as EPA and DHA)) via orogastric feeding tube and six control rats were given 0.25 cc water, both enterally. The emulsion was made as described in Example 1. The rats recovered for two hours, and then underwent hypoxia-ischemia for 65 minutes of 8% oxygen at a constant temperature. The six pre-treated rats were given another dose of 50 mg omega-3 triglyceride emulsion immediately after the hypoxia-ischemia and control rats were given 0.25 cc water. All rats were euthanized at 72 hours of reperfusion. The brains were removed and cut into 2 mm sections and stained with 2, 3, 5, Triphenyl-2H-tetrazolium chloride (TTC). The sections were scored as follows:

0—no evidence of edema or cell death

1—edema without cell death

2—edema with minimal cell death

3—edema with significant cell death.

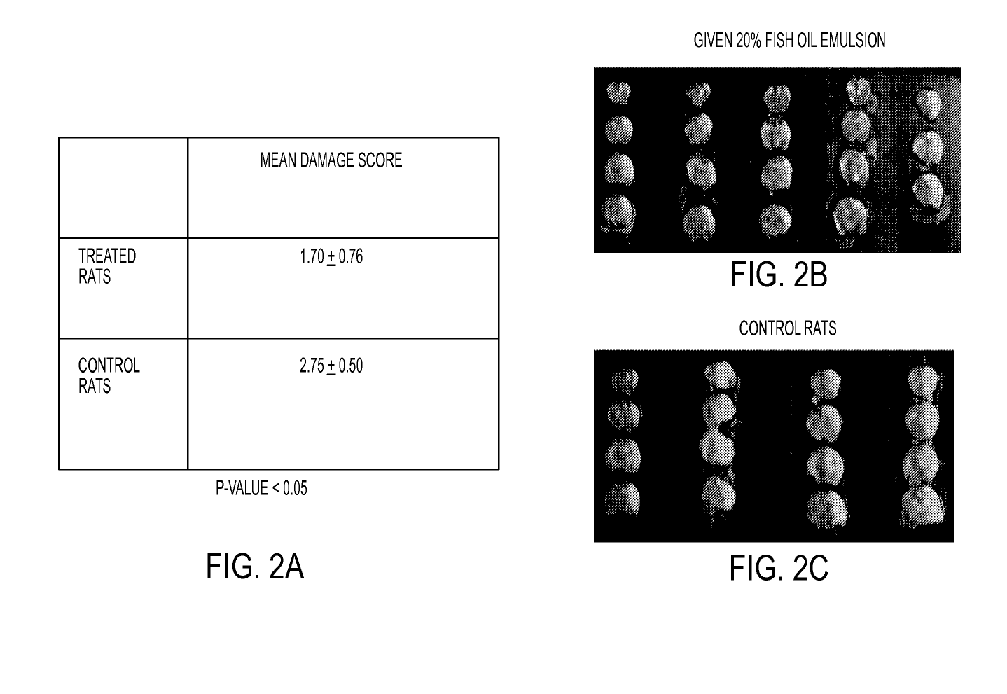

The 65 minutes of hypoxia-ischemia produced damage in all rats. Four of the six control rats survived with a mean score of 2.75+/−0.50, while five of the six treated rats survived with a mean score of 1.70+/=0.76 (p<0.05). FIG. 2.

### Example 3

### Treatment of Rats with Omega-3 Triglyceride Lipid Emulsion Prior to 60 Minutes of Hypoxia

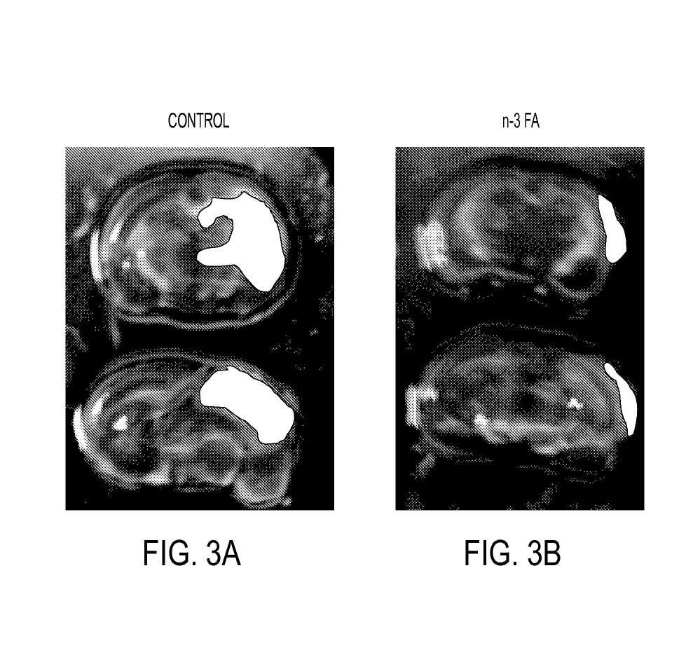

Postnatal day 19-21 Wistar rats were subjected to unilateral (right) carotid artery. Immediately after ligation, six rats were given 50 mg of a 20% omega-3 triglyceride emulsion (0.25 cc), and six control rats were given 0.25 cc water, both enterally. The emulsion was as described above in Example 1. Rats were allowed to recover for two hours, and then underwent hypoxia-ischemia for 60 minutes of 8% oxygen at a constant temperature. The six pre-treated rats were given another dose of 50 mg omega-3 triglyceride lipid emulsion immediately after the hypoxia/ischemia and control rats were given 0.25 cc water. At 72 hours of reperfusion, the rats were euthanized and their brains removed, cut into 2 mm sections and stained with 2,3,5 triphenyl-2H-tetrazolium chloride (TTC). FIG. 3 The damage in each animal was then given a score from 0 (no damage) to 4 (>60% ipsilateral hemisphere infarcted). All of the vehicle-treated animals suffered brain damage, with a mean damage score of 2.00+0.89; the omega-3 triglyceride lipid emulsion-treated rats were significantly less damaged, having a mean damage score 0.33+0.52, p<0.05. The size of brain infarcts was determined by TTC staining.

These results show that when omega-3 triglycerides were administered either immediately before and/or after hypoxia-ischemia they confer a significant neuroprotection. Very similar results were obtained when the omega-3 triglycerides were injected parenterally.

### Example 4

### Treatment Following Hypoxic Ischemia

Post-natal day 19-21 rat pups were subjected to unilateral carotid artery ligation and 60 minutes of hypoxic ischemia, according to the previously described protocol. On four separate occasions, rats were treated by parenteral injection of omega-3 triglyceride emulsion (100 mg) immediately after the insult, and again at four hours after the insult. The emulsion was as described above in Example 1. Brain damage was evaluated by TTC staining at 72 hours of reperfusion. In each instance, administration of the omega-3 triglyceride oil emulsion provided greater than 50% protection, i.e. reduction of tissue damage.

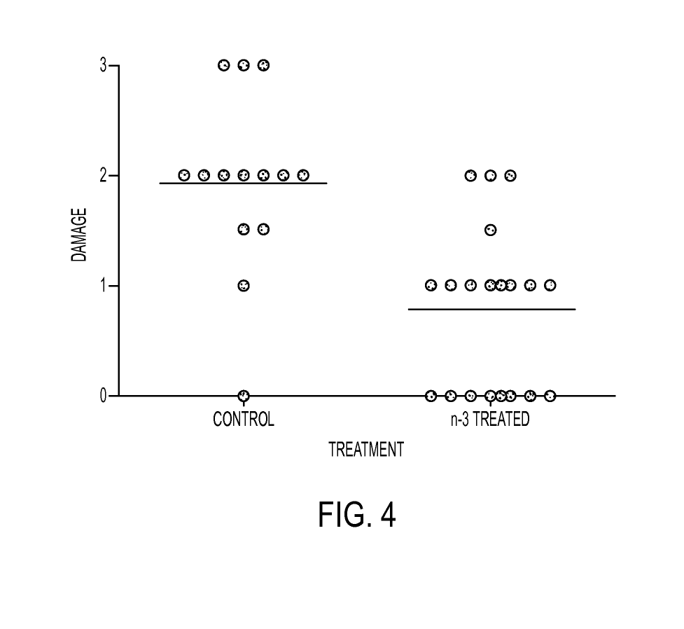

FIG. 4 shows the results of these experiments. FIG. 4 represents at total of 14 control subjects (saline-treated) and 21 treated subjects (omega-3 triglyceride emulsion treated). Mean damage scores were: 1.93±0.22 (SEM), control, 0.78±0.16 emulsion-treated; p<0.0001 by two-tailed test. Thus, in addition to the significance of the overall protection, it can be seen that 40% of the treated animals were 100% protected (no damage at all, compared to 1/14 untreated; 40% suffered only mild damage, compared to 1/14 mildly damaged untreated animals. These results indicate that treatment following hypoxic-ischemia provides a neuroprotective benefit as indicated by a reduction of tissue damage.

Preliminary experiments conducted in the adult mouse show a comparable level of neuroprotection from hypoxic-ischemic damage.

Fatty acyl composition analyses of brain lipids (by gas liquid chromatography) after hypoxia/ischemia show no relative differences between infarcted brain versus non infarcted brain indicating that effects of acute administration of omega-3 emulsions are not dependent on fatty acid compositional changes in brain membranes. In the infarcted areas, however, absolute concentrations of all fatty acids fell to similar degrees by about 15% (μg fatty acid per gram wet brain) indicating brain edema. This decrease did not occur with administration of omega-3 emulsions indicating that these omega-3 fatty acids prevented the brain edema as well as infarction.

### Example 5

### Materials and Methods for Studying Cerebral Hypoxia-Ischemia

### Ethics Statement

All research studies were carried out according to protocols approved by the Columbia University Institutional Animal Care and Use Committee (IACUC) and in accordance with the Association for Assessment and Accreditation of Laboratory Animal Care guidelines.

### Materials

Pure Tri-DHA and Pure Tri-EPA shown in Example 7 were purchased from Nu-Chek Prep, Inc. (Elysian, Minn.). Egg yolk phosphatidylcholine was obtained from Avanti Polar-Lipids, Inc. (Alabaster, Ala.).

### Lipid Emulsions

Four different types of lipid emulsions were used in Example 6 and Example 8. Omega-3 fish oil-based and n-6 soy oil-based emulsions were commercially prepared intravenous phospholipid-stabilized emulsions, and the “n-3 TG” formula (TG90/DHA30) is set forth in Example 5 and Table 1 and has been previously described^{13,18}. The n-6 TG emulsions were produced from soybean oil rich in n-6 FA: linoleic acid constituting about 55% of total FA. For doses of injected n-3 TG emulsions in the examples, it was determined that the amount administered contained ˜50% of the TG-FA as DHA (30%) and EPA (28%). Thus, 1 gm of the n-3 TG emulsions is expressed as 0.5 gm n-3 TG.

Other emulsions having pure (99%) DHA or pure (99%) EPA was also tested as described; these emulsions ha 90% triglyceride of which 99% was DHA or EPA. For doses of injected “n-3 TG” (Table 1) emulsions, an amount was calculated to achieve an administration containing 50% of the TG-FA as DHA and EPA (Table 1). Thus, 1 gm of TG emulsions is expressed as 0.5 gm n-3 TG.

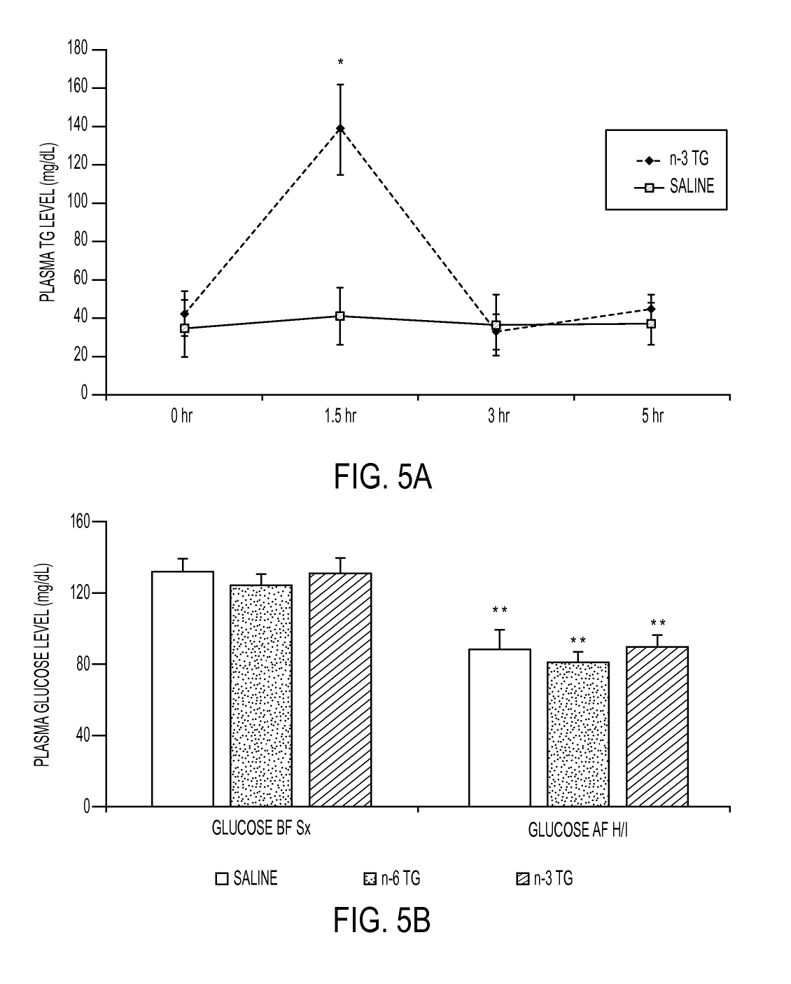

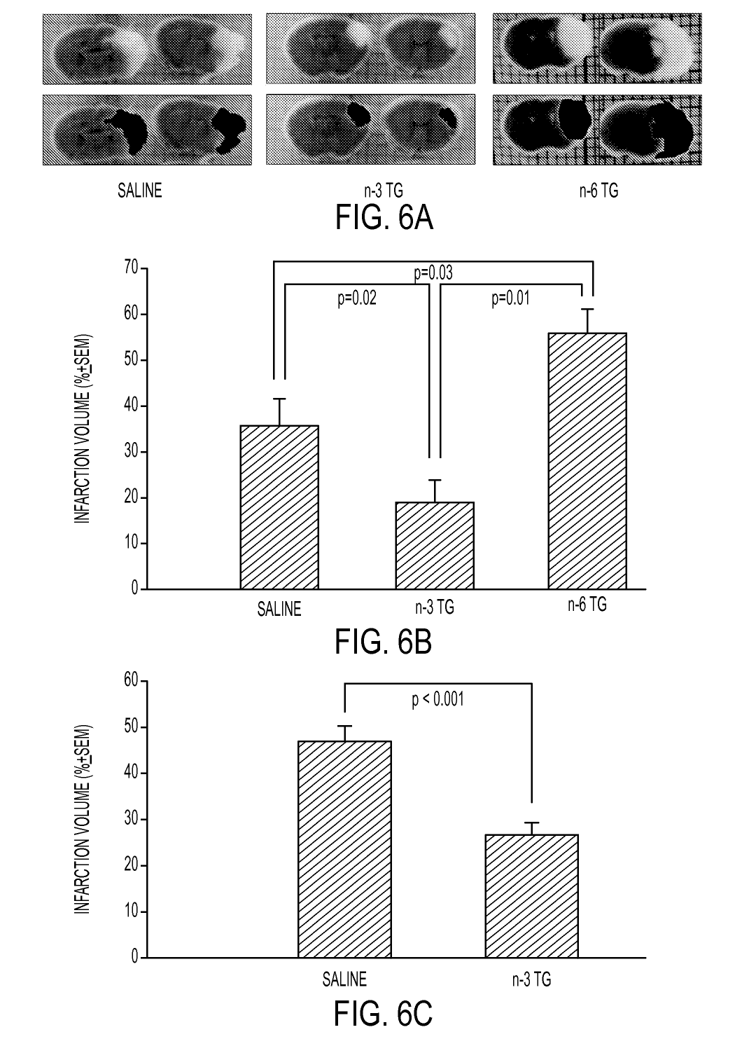

The n-6 TG emulsions described in Table 1, right column and shown in FIG. 5B and FIG. 6B, and comprise 20% omega-6 oil (n-6) by weight in grams per 100 ml of emulsion, 0% DHA, 0% EPA and 55% TG from linoleic acid (Table 1, right column). The n-6 TG emulsions were produced from soy bean oil rich in n-6 FA: linoleic acid constituting about 55% of total FA.

TABLE 1
Fatty Acid Composition of Triglyceride Lipid Emulsions¹

| | “n-3 TG” (g/100 ml) | |
|---|---|---|
| | (also referred to as | n-6 TG |
| Source | “n3-TG90-DHA30”) | (g/100 ml) |
| | g/100 mL | g/100 mL |
| Soybean oil | — | 20 |
| Fish oil | 10 | — |
| Egg phosphatidylcholine | 1.2 | 1.2 |
| Glycerol | 2.5 | 2.25 |
| FA (% of total FA) | % | % |
| Palmitic acid (C16:0) | 2.5-10 | 7-14 |
| Stearic acid (C18:0) | 0.5-2 | 1.4-5.5 |
| Oleic acid (C18:ln-9) | 6-13 | 19-30 |
| Linoleic acid (C18:2n-6) | 1-7 | 44-62 |
| Arachidonic acid (C20:4n-6) | 1-4 | <0.5 |
| α-linolenic acid (C18:3n-3) | 2 | 4-11 |
| Eicosapentaenoic acid | 12.5-28.2 | — |
| (C22:6n-3) | | |
| Docosahexaenoic acid | 14.4-30.9 | — |
| (C22:6n-3) | | |

¹Data provided by Fresenius Kabi AG; FA, Fatty acids.

The pure Tri-DHA (99% DHA) and Tri-EPA (99% EPA) emulsions in Example 7 were VLDL-sized and laboratory-made with TG oil and egg yolk phospholipid using sonication and centrifugation procedures that are known in the art.^{3,4} Briefly, 200 mg Tri-DHA (Tri-DHA oil>99%) or Tri-EPA (Tri-EPA oil>99%) was mixed with a 5:1 weight ratio of egg yolk phosphatidylcholine (40 mg). The mixture was fully evaporated under N₂ gas, and was further desiccated under vacuum overnight at 4° C. The dried lipids were resuspended in 1 mL of lipoprotein-free buffer (LPB) (150 mmol/L NaCl, 0.5 ml of 0.1% glycerol and 0.24 mmol/L EDTA, pH 8.4, density 1.006 g/mL) at 60° C. with added sucrose (100 mg/l mL LPB) to remove excess phospholipid liposomes. The lipid emulsions were then sonicated for 1 hr at 50° C., 140 W under a stream of N₂ using a Branson Sonifier model 450 (Branson Scientific, Melville, N.Y.). After sonication, the solution was dialyzed in LPB for 24 hr at 4° C. to remove sucrose. The final emulsions comprising VLDL-sized particles were analyzed for the amount of TG and PL by enzymatic procedure using GPO-HMMPS, glycerol blanking method (Wako Chemicals USA, Inc., Richmond, Va.) and choline oxidase-DAOS method (Wako Chemicals USA, Inc., Richmond, Va.). The TG:phospholipid mass ratio was 5.0±1.0:1 similar to that of VLDL-sized particles. The emulsions were then stored under argon at 4° C. and were used within two weeks of preparation.

### Methods

### Induction of Unilateral Cerebral H/I

Three-day-old C57BL/6J neonatal mice of both genders were purchased from Jackson Laboratories (Bar Harbor, Me.) with their birth mother. The Rice-Vannucci model of H/I was used and modified to p10 neonatal mice.⁵ Briefly, on postnatal day 10 H/I was induced by the ligation of the right common carotid artery, which was further cauterized and cut under isoflurane anesthesia. The investigator was blinded to the lipid emulsion treatment during the surgery and after the surgery. The entire surgical procedure was completed within 5 min for each mouse. Pups were then allowed to recover with their dams for 1.5 hr. Surrounding temperature during experiments was kept at 28° C. Mice were then exposed to systemic hypoxia for 15 min in a hypoxic chamber in a neonatal isolette (humidified 8% oxygen/nitrogen, Tech Air Inc., White Plains, N.Y.).⁵ The ambient temperature inside the chamber during hypoxia was stabilized at 37±0.3° C. To minimize a temperature-related variability in the extent of the brain damage, during the initial 15 hr of reperfusion mice were kept in an isolette at the ambient temperature of 32° C.

### Quantification of Brain Infarction

After 24 hr of reperfusion, the animals were sacrificed by decapitation and brains were immediately harvested. 1-mm coronal slices were cut by using a brain slicer matrix. Slices were then immersed in a PBS solution containing 2% triphenyl-tetrazolium chloride (TTC) at 37° C. for 25 min. TTC is taken up into living mitochondria, which converts it to a red color.⁶ Thus, viable tissue stains brick-red, and nonviable (infarcted) tissue can be identified by the absence of staining (white). Using Adobe Photoshop and NIH Image J imaging applications, planar areas of infarction on serial sections were summed to obtain the volume (mm³) of infarcted tissue, which was divided by the total (infarcted+non-infarcted) volume of the hemisphere ipsilateral to carotid artery ligation, and expressed as a percentage of total volume.

### Experimental Groups

H/I brain injury was induced in different groups of animals, which received specific treatments before and after H/I injury. Animals followed different treatment protocols.

### Protocol 1: Pre-H/I Treatment of n-3 TG or n-6 TG Emulsions

Two doses of n-3 TG or n-6 TG emulsions or vehicle (saline, equal volumes/kg) were administered to non-fasting rodents at a fixed dose of 3 mg of n-3 TG or n-6 TG-FA per mouse for each injection (equivalent to a maximum of 1.5 g of total TG/kg; p10 mice weighed 4-6 gm for these experiments). The first dose was i.p. administered immediately after surgery, and the second immediately at the end of the 15 min hypoxic period. Volumes injected for TG emulsions and saline were always equal.

Since the n-3 emulsions of the invention contain low concentrations of alpha-tocopherol as an anti-oxidant agent, in separate experiments an equivalent dose of pure alpha-tocopherol (Vital E®, Intervet, Schering Plough) to match the content in the n-3 emulsion (0.8 g/L) was given to neonatal mice by i.p. injection of at a dose of 5 mg alpha-tocopherol/kg body weight; the same amount contained in each i.p. injection of the n-3 TG emulsions.

Protocol 2: Post-H/I Treatment with “n-3 TG”

Two doses of the commercially available n-3 TG emulsion (Table 1) or saline were i.p. injected into non-fasting rodents at 0.75 g of n-3 TG/kg body weight for each dose (equivalent to 1.5 g of total TG/kg). The first dose was administered immediately after 15-min hypoxia, and the second at 1 hr after start of the reperfusion period.

### Protocol 3: Dose Response, Timing and Specificity of “n-3 TG”

Two types of omega-3-containing lipid emulsions, either Tri-DHA or Tri-EPA (0.1 g n-3 TG/kg or 0.375 g n-3 TG/kg body weight for each dose), were administered twice to non-fasting rodents according to the amount of DHA and EPA in the n-3 TG emulsions. See Table 1. The first dose was initially administered immediately after 15-min hypoxia, and the second after 1 hr of reperfusion. Then in different sets of experiments, the efficacy of Tri-DHA emulsions was determined, with the initial injection administered at four-time points (0 hr, or at 1 hr, 2 hr or 4 hr after H/I), 0.375 g n-3 TG/kg body weight for each dose. For the immediate treatment of 0 hr, the first dose was injected immediately after 15-min hypoxia, with a second injection after 1 hr of reperfusion, whereas in the “delayed” treatments, the first dose was given after the 1^{st} or 2^{nd} or 4^{th} hr of reperfusion and a second dose was administered 1 hr after the 1^{st} dose.

### Measurement of Blood TG and Glucose Levels

Blood samples taken directly from left ventricle of hearts under isoflurane inhalation were obtained from a separate cohort of non-fasting, 10-day-old mice. Samples were taken over a 5-hr period after a single i.p. injection of either 0.75 g n-3 TG/kg commercially available emulsions or saline. Total plasma TG was enzymatically measured by GPO-HMMPS, glycerol blanking method (Wako Chemicals USA, Inc., Richmond, Va.). For glucose levels, blood samples were taken from mouse tails from a separate cohort of non-fasting 10-day-old mice. Samples were taken at two time points from each mouse. The first sample was taken at time zero before surgery and n-3 TG injection, and the second at about 10 min after H/I and n-3 TG injection (approximately 100 min after surgery as described under the Unilateral Cerebral H/I protocol above). Blood glucose levels were electrochemically measured in mg/dL by a glucose meter (OneTouch Ultra, LifeScan, Inc., Milpitas, Calif.).

### Measurement of Cerebral Blood Flow (CBF) by Laser Doppler Flowmetry (LDF)

In a cohort of neonatal C57BL/6J mice pups subjected to carotid artery ligation and recovery as described above, relative CBF was measured during hypoxia in ipsilateral (right) hemispheres using a laser Doppler flow meter (Periflux 5000). In these mice, in preparation for CBF measurement the scalp was dissected under isoflurane anesthesia and Doppler probes were attached to the skull (2 mm posterior and 2 mm lateral to the bregma) using fiber optic extensions. Only local anesthesia (1% lidocaine) was used postoperatively. Mice were then placed into a hypoxia chamber (8% O₂/92% N₂). Changes in CBF in response to hypoxia were recorded for 20 min and expressed as percentage of the pre-hypoxia level for n-3 treated and saline treated neonatal mice.

Measurement of Bleeding Time after “n-3 TG” Injection

Bleeding times were measured in mice after severing a 3-mm segment of the tail.⁷ Two doses of saline were administered vs. n-3 TG in a similar time frame as the original protocol: an initial injection followed by a second injection at 2 hr later. Bleeding times were measured at 45 min after the second dose. The amputated tail was immersed in 0.9% isotonic saline at 37° C., and the time required for the stream of blood to stop was defined as the bleeding time. If no cessation of bleeding occurred after 10 min, the tail was cauterized and 600 s was recorded as the bleeding time.

### Long-Term Assessment of Brain Tissue Death

A long-term assessment of cerebral injury was performed at 8 wk. after neonatal H/I insult. This cohort of mice at p10 underwent unilateral H/I followed by post H/I injections with either 0.375 g Tri-DHA/kg (n=6) or saline (n=5) as described above. At 8 wk. after H/I, mice were sacrificed by decapitation. Brains were removed, and embedded in Tissue Tek-OTC-compound (Sakura Fineteck, Torrance, Calif.) with subsequent snap freezing in dry ice-chilled isopentane (−30° C.), and stored at −80° C. For analysis, coronal sections (10 μm every 500 μm) were cut serially in a Leica cryostat and mounted on Superfrost slides (Thermo Scientific, Illinois). Sections were processed for Nissl staining by using Cresyl Violet Acetate (Sigma-Aldrich, St. Louis, Mo.). Using Adobe Photoshop and NIH Image J imaging applications, 9 sections from each brain containing both the right and left hemispheres were traced for brain tissue area. As previously described⁸ the area of left control or contralateral hemisphere which had not had injury was given a value in 100% for each animal. The brain area remaining in the right injured ipsilateral hemisphere was then compared to the left hemisphere, and the difference was taken as the percent right brain tissue loss, for each animal.

### Statistical Analysis

Data are presented as mean±SEM. Plasma TG levels were compared at each time point after i.p. injection of n-3 TG emulsion. Student t tests were used for 2-group comparisons. 1-way ANOVA, followed by Bonferroni procedure for post hoc analysis to correct for multiple comparisons, was used to compare the differences among the emulsions on the infarct areas across coronal sections. Statistical significance, which was analyzed by using SPSS software 16.0 (SPSS Inc., Chicago, Ill.), was determined at p<0.05.

### Example 6

### n3 TG Emulsions are Neuroprotective after Cerebral Hypoxic-Ischemic Injury in Neonatal Mice

### Effects of “n-3 TG” on Blood Triglyceride and Glucose Levels, and Bleeding Time

To determine if triglyceride (TG) from the n-3 TG emulsions was systemically absorbed, the blood TG levels were examined up to 5 hr after i.p. injection. After n-3 TG injection, there was a substantial increase of TG levels up to three fold higher at 1.5 hr (p<0.05) compared to the baseline, followed by a decrease of levels to baseline at 3 and 5 hr. See FIG. 5A. This indicates that n-3 TG entered into the blood stream and was being catabolized. In comparison, TG levels of saline-injected mice remained constant over the 5-hr time period reflecting normal blood TG levels in neonates.

After H/I, blood glucose levels might affect infarct size.⁹ Therefore, blood glucose levels were measured in each group (n-3 TG vs. n-6 TG vs. saline control) prior to surgery and after 15-min H/I after TG or saline injection. See FIG. 5B. No difference in blood glucose levels among groups was observed when comparing at the same time point. Still, after H/I insult, blood glucose levels decreased similarly, about 30% or more, in all groups (p<0.05).

There was no difference in capillary bleeding times in n-3 treated mice (437±82 sec) as compared to saline controls (418±90 sec).

“n-3 TG” Did not Change Cerebral Blood Flow after H/I

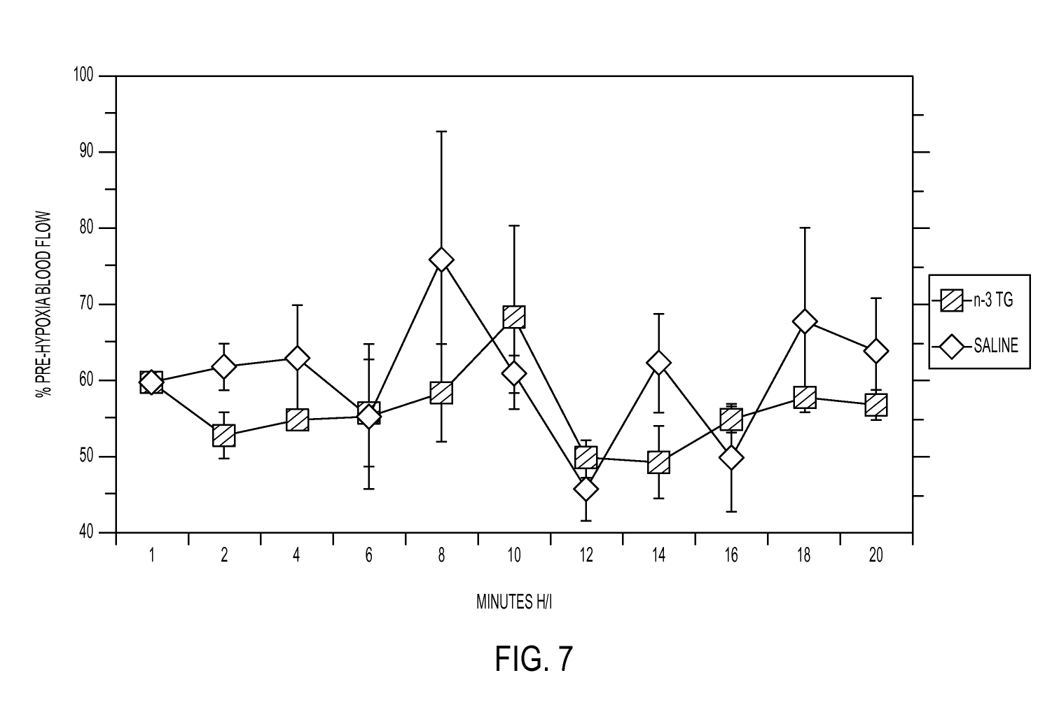

There was no effect on CBF in the ipsilateral hemisphere of n-3 treated neonatal mice as compared to saline treated animals. Immediately after right common carotid artery ligation, and initiation of hypoxia at 8% oxygen, CBF was approximately 25% of initial (pre-H/I) level in the ipsilateral hemisphere in both control and n-3 treated groups, and this was maintained for the duration of hypoxia. In the contralateral (unligated) hemisphere blood flow was unchanged in both groups. Very similar blood flow levels were maintained in neonatal H/I mice whether they were saline treated or n-3 treated in this model. See FIG. 7.

### “n-3 TG” but not “n-6 TG” Protects Brain Against H/I Injury

Coronal sections of brains were stained with TTC to quantify the extent of post H/I brain injury and the effect of n-3 TG injection. See FIG. 6. FIG. 6A shows representative images of neonatal mouse brain from saline treated, n-6 TG emulsion treated and n-3 TG emulsion treated mice with pre-and-post injection after H/I, respectively. In all H/I animals, tissue death was localized to the right hemisphere (ipsilateral to ligation) as illustrated by the white areas in the upper panels of FIG. 6A. The image in the lower panels, FIG. 6A, demonstrated tracings of the infarcted areas for quantifying infarct volume using NIH Image J. The brains from saline treated animals exhibited a consistent pannecrotic lesion involving both cortical and subcortical regions ipsilateral to the ligation. In the majority of the animals the neuroprotection after n-3 TG injection was most marked in the subcortical area, whereas saline treated mice had large cortical and subcortical infarcts. See FIG. 6A.

Infarct volume was substantially decreased in n-3 TG treated mice (n=28) compared to saline treated littermates (control) (n=27), 19.9±4.4% vs. 35.1±5.1%, respectively (p=0.02). See FIG. 6B. There was a significant increase in infarct volume with n-6 TG emulsion injection compared to saline control (p=0.03) and the n-3 TG groups (p<0.01).

Because alpha-tocopherol is a component of the TG emulsions (present in low concentrations to prevent FA oxidation), TTC staining was used to compare the extent of cerebral H/I injury in alpha-tocopherol treated and saline treated neonatal mice. There was no significant difference in infarct volume between brains in alpha-tocopherol injected mice compared to saline treated mice (data not shown).

It was then determined whether n-3 TG were effective if injected only after H/I (without injection prior to H/I. See FIG. 6C. Similarly, the smaller n-3 TG associated lesions were mainly subcortical (data not shown). Compared to saline controls in the immediate post-H/I treatment the total infarct area was significantly reduced almost 50% in the n-3 TG post H/I treated group.

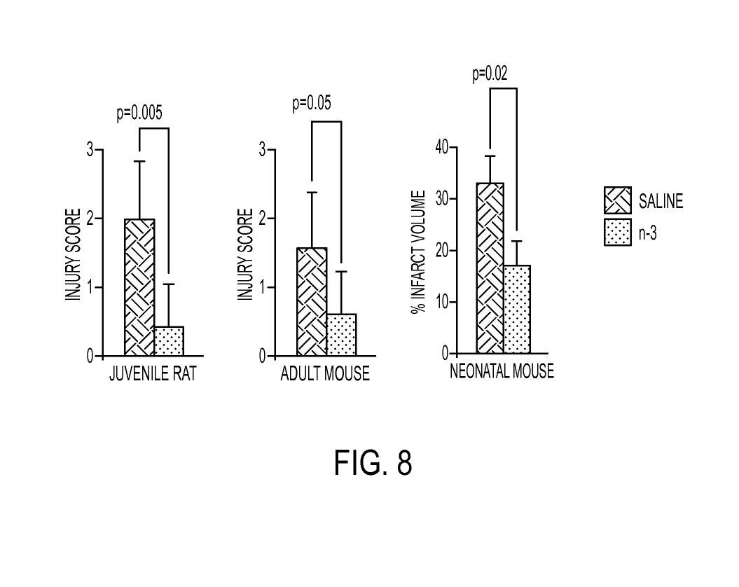

Additional experiments were done to study the effect of administering the n-3 TG emulsions on cerebral hypoxia-ischemia, the results of which are shown in FIGS. 8-11. Animals were handled and treated as described as described above. TTC staining showed that cerebral infarct size decreased after i.p. injections (FIG. 8) in three different rodent models including, the juvenile mouse, the adult mouse, and the neonatal mouse. Each injection provided about 15 mg DHA per mouse, or about 500 mg DHA/Kg.

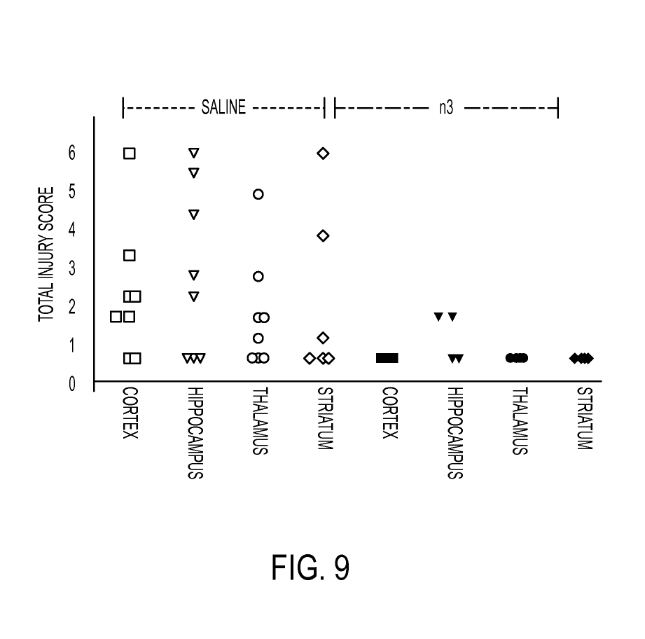

FIG. 9 shows that n-3 TG attenuated brain injury after hypoxia/ischemia. Using the protocol described in Example 5, it was shown that injection of n-3 TG markedly protected different regions of the brain from stroke injury after hypoxia/ischemia. Each injection provided about 15 mg DHA per mouse, or about 500 mg DHA/Kg. Other unpublished data show that n-3 TG also reduced 3-Nitrotyrosine protein oxidation and 4-Hydroxynonenal lipid peroxidation 24 hours after hypoxia/ischemia.

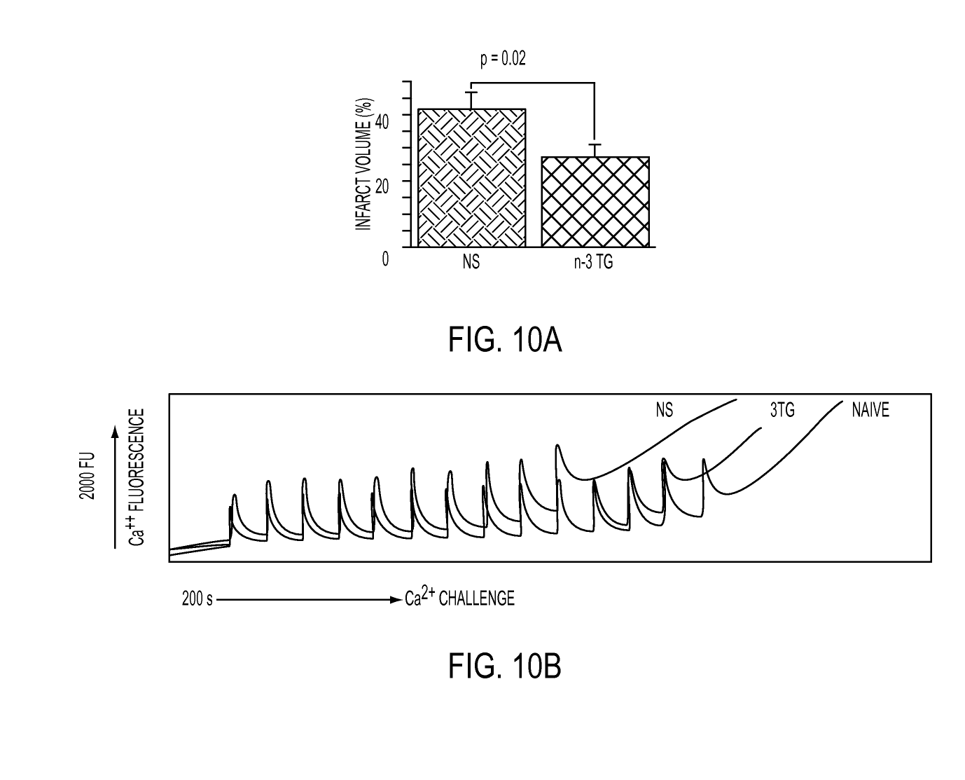

FIG. 10A-10B shows that i.p. injection n-3 TG after hypoxia/ischemia reduced infarct volume which correlated with maintenance of the integrity of the mitochondrial membrane as is evidenced by a decrease in brain Ca²⁺⁻ induced opening of mitochondrial permeability transition pores (mPTP) that typically occurs in untreated animals after hypoxia/ischemia. Each injection provided about 15 mg DHA per mouse, or about 500 mg DHA/Kg.

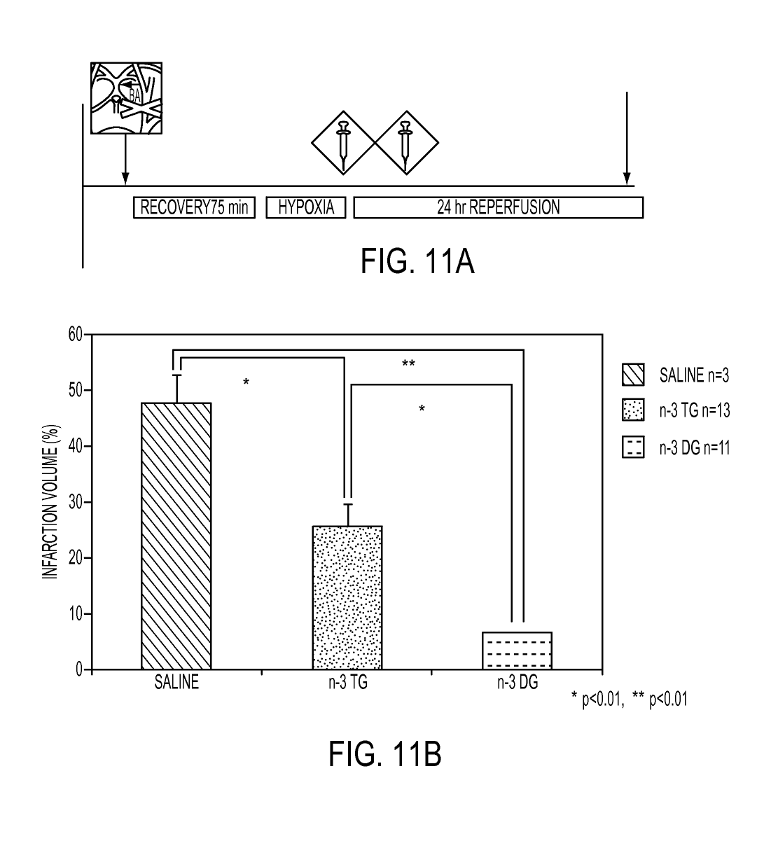

FIG. 11A-11B show that both n-3 TG and n-3 DG (diglyceride) emulsions reduce cerebral infarct size.

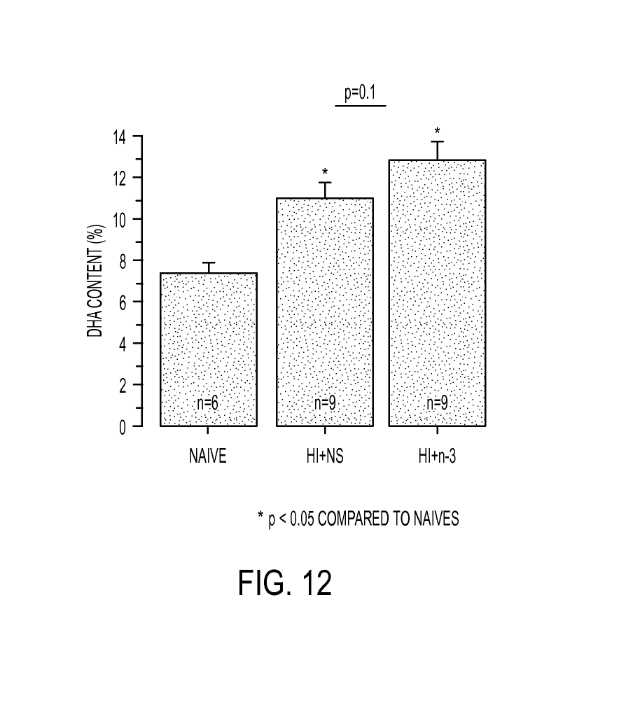

FIG. 12 illustrates DHA content in isolated brain mitochondria. These data show that at 4 hours after injection of n-3 TG after hypoxia/ischemia-induced stroke that the DHA content in brain mitochondria is increased. It is speculated that this increase likely contributes to the beneficial effects of DHA. It is noted that EPA content was not increased in brain mitochondria (Data not shown). Each injection provided about 15 mg DHA per mouse, or about 500 mg DHA/Kg.

### REFERENCES CITED IN EXAMPLE 5 AND EXAMPLE 6

1. Oliveira F L, Rumsey S C, Schlotzer E, Hansen I, Carpentier Y A, et al. (1997) Triglyceride hydrolysis of soy oil vs fish oil emulsions. JPEN J Parenter Enteral Nutr 21: 224-229.
2. Qi K, Seo T, Al-Haideri M, Worgall T S, Vogel T, et al. (2002) Omega-3 triglycerides modify blood clearance and tissue targeting pathways of lipid emulsions. Biochemistry 41: 3119-3127.
3. Qi K, Al-Haideri M, Seo T, Carpentier Y A, Deckelbaum R J (2003) Effects of particle size on blood clearance and tissue uptake of lipid emulsions with different triglyceride compositions. JPEN J Parenter Enteral Nutr 27: 58-64.
4. Schwiegelshohn B, Presley J F, Gorecki M, Vogel T, Carpentier Y A, et al. (1995) Effects of apoprotein E on intracellular metabolism of model triglyceride-rich particles are distinct from effects on cell particle uptake. J Biol Chem 270: 1761-1769.
5. Ten V S, Bradley-Moore M, Gingrich J A, Stark R I, Pinsky D J (2003) Brain injury and neurofunctional deficit in neonatal mice with hypoxic-ischemic encephalopathy. Behav Brain Res 145: 209-219.
6. Liszczak T M, Hedley-Whyte E T, Adams J F, Han D H, Kolluri V S, et al. (1984) Limitations of tetrazolium salts in delineating infarcted brain. Acta Neuropathol 65: 150-157.
7. Denis C, Methia N, Frenette P S, Rayburn H, Ullman-Cullere M, et al. (1998) A mouse model of severe von Willebrand disease: defects in hemostasis and thrombosis. Proc Natl Acad Sci USA 95: 9524-9529.
8. Seo T, Blaner W S, Deckelbaum R J (2005) Omega-3 fatty acids: molecular approaches to optimal biological outcomes. Curr Opin Lipidol 16: 11-18.
9. Bruno A, Biller J, Adams H P, Jr., Clarke W R, Woolson R F, et al. (1999) Acute blood glucose level and outcome from ischemic stroke. Trial of ORG 10172 in Acute Stroke Treatment (TOAST) Investigators. Neurology 52: 280-284.

### Example 7

### DHA but not EPA is Neuroprotective after H/I

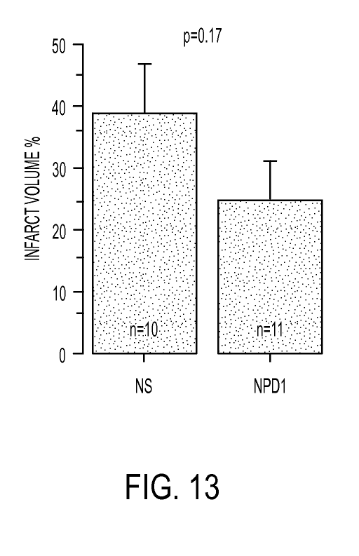

FIG. 13 shows that cerebral infarct volume at 24 hr post hypoxia/ischemia was reduced in mice post-treated with neuroprotectin D1 (NPD1) (20 ng) which is a catabolic product of DHA, as opposed to treatment with saline vehicle). In addition, this product of DHA also maintains close to normal brain mitochondrial permeability in isolated mitochondria exposed to high oxygen or after hypoxic-ischemic injury in mice. Note that while NPD1 did have beneficial effects, it was not as effective as treatment with omega-3 triglyceride emulsions with pure (99%) DHA. Dosage of NPD1 was 20 ng (nanogram) per mouse.

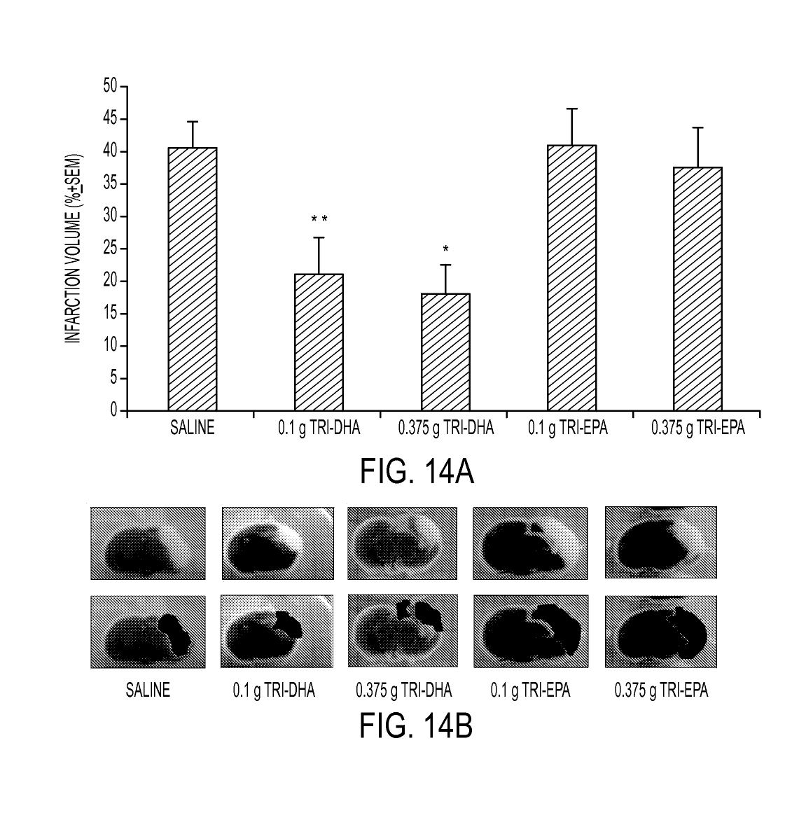

To determine possible differences in neuroprotection of EPA vs. DHA, the extent of brain injury was studied using the post-H/I treatment protocol with pure (99%) Tri-DHA vs. Tri-EPA in two dosages (0.1 g TG/kg vs. 0.375 g TG/kg). Pure TG was made as described in Example 5. No statistical differences in brain infarct volume between 0.1 g TG/kg and 0.375 g TG/kg Tri-DHA treated groups were observed. However, compared to saline control, total infarct size was reduced by a mean of 48% and 55% by treatment with 0.1 and 0.375 g TG/kg Tri-DHA, respectively. See FIG. 14A-14B. Neuroprotection was not observed with Tri-EPA injection at either of the two doses compared with saline treatment.

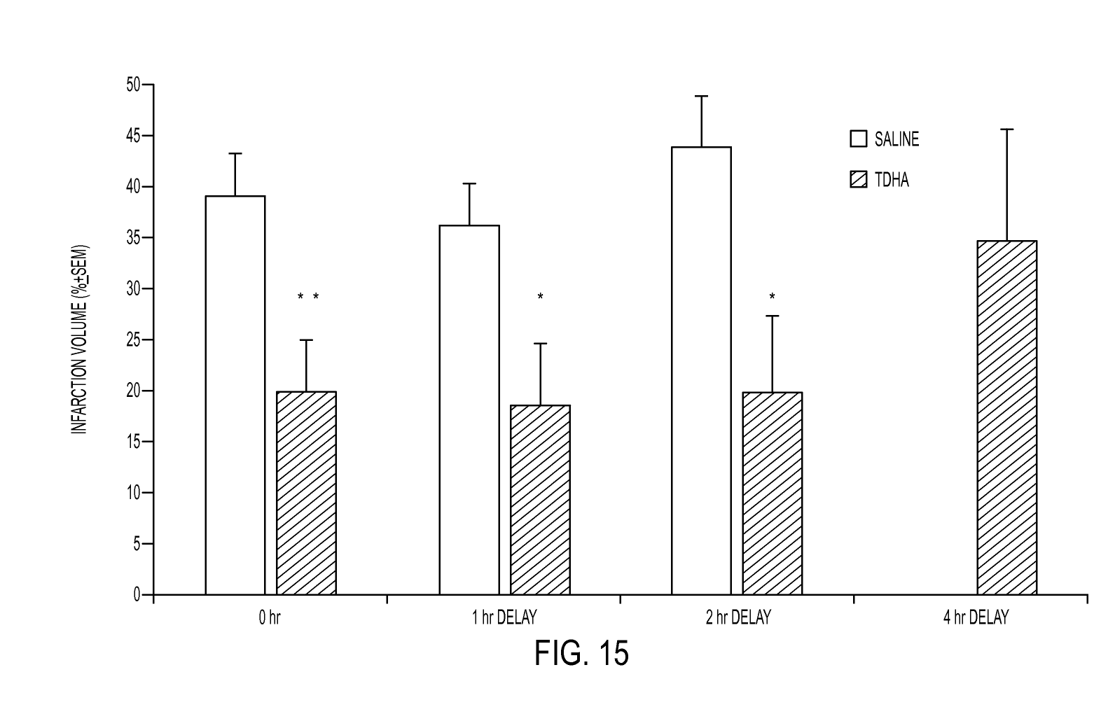

To better approximate realistic timelines for neuroprotection after stroke for humans delayed treatment protocols were performed in an effort to study the therapeutic window of Tri-DHA emulsions. No protective effect from Tri-DHA after a 4-hr delay in treatment was noted compared with saline group. However, Tri-DHA administered at 0 hr immediately post H/I, and then again at 1-hr and 2-hr post stroke showed similar reduced (˜50%) brain infarct volumes compared to saline treated animals. See FIG. 15. This substantial protection occurred mainly in subcortical areas similar to the findings described above.

### Long-Term Neuroprotection

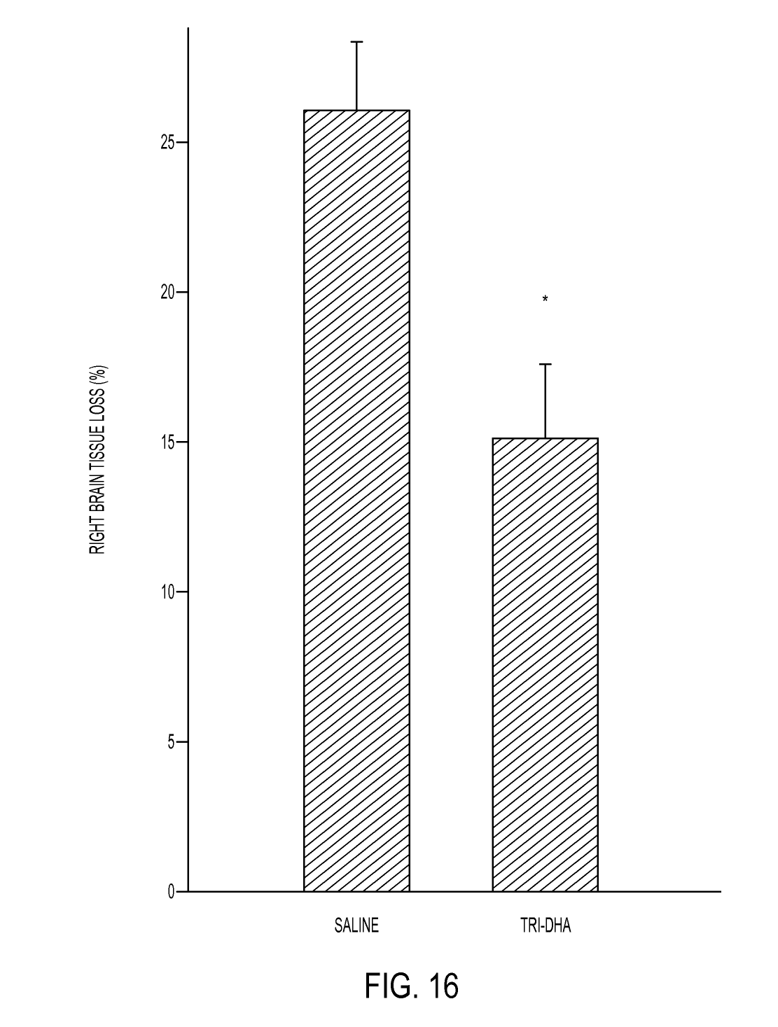

Coronal brain sections of adult mice were processed for Nissl staining (FIG. 16) to examine the effects of H/I and Tri-DHA treatment on brain and neuronal cell loss for long-term outcome at 8 wk. after H/I insult. As compared to the left control (contralateral hemisphere), the injured areas of the right hemisphere display gross neuronal cell loss. As shown in FIG. 16, brain tissue loss was markedly increased by 1.67 fold in the right hemisphere of saline-treated mice (n=5) as compared to Tri-DHA treated mice (n=6), 25.0±2.4% vs. 15.0±2.5%, respectively (p=0.02). Thus, neuroprotection after injury and Tri-DHA injection that are observed 24 hr after H/I is maintained 8 weeks later, without further injections of omega-3 triglycerides.

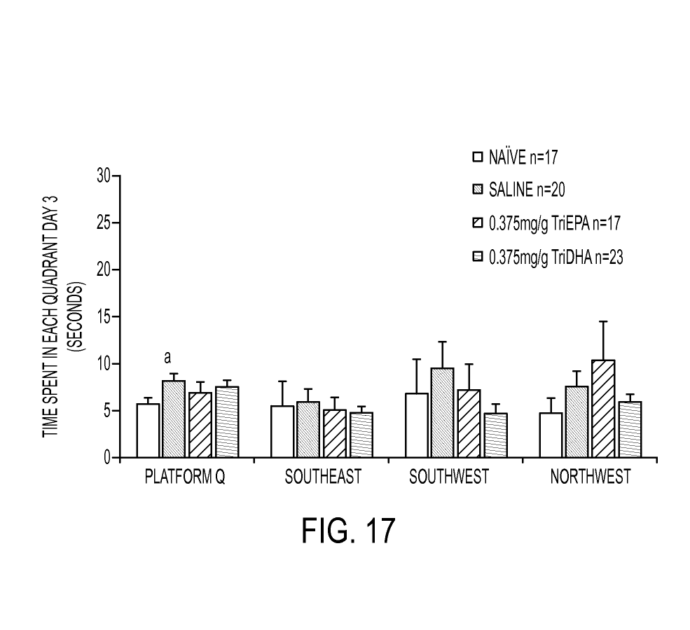

Further experiments were conducted to optimize the efficacy of n-3 TG emulsion on treating damage from cerebral hypoxia/ischemia. First the effect of administering pure Tri-DHA (99% DHA) and Tri-EPA (99% EPA) emulsions were VLDL-sized and laboratory-made with TG oil and egg yolk phospholipid as described in Example 5, using both n-3TG emulsions as well as Tri-DHA and Tri-EPA emulsions. In these experiments only Tri-DHA and Tri-EPA emulsions were tested for an 8-week follow-up, and were administered to test memory and neurologic function. First, navigational memory was assessed 8 weeks after mice were subjected to hypoxia/ischemia to cause a stroke as an indicator of neurofunction following treatment with either 99% pure DHA or pure 99% EPA. Navigational memory was recorded 8 weeks after the initial injury on day 3 of the water-maze testing period, on which the platform was removed. FIG. 17 shows the time spent in each quadrant. The results show that navigation memory and neurofunction were maintained and in fact were significantly enhanced in mice that had been treated twice (both immediately after the hypoxic-ischemic injury and one hour later) with n-3 TG having pure (99%) DHA. Unexpectedly, it was discovered that the neurologic performance results were significantly better with an omega-3 emulsion having pure DHA than with pure EPA, although EPA seemed to have no deleterious effects. Mice were given 375 mg of n-3 TG 99% DHA or 99% EPA per Kg immediately after hypoxic-ischemic injury and 1 hour later, 8 weeks before testing was done.

### Example 8

### Omega-3 Triglyceride Emulsions (n-3 TG; TG90/DHA30) Reduced Ex Vivo Cardiac Hypoxia-Ischemia Reperfusion Damage

### Experiments

### Materials and Methods: Animal Care

All studies were performed with the approval of the Institutional Animal Care and Use Committee at Columbia University, New York University School of Medicine, and conform to the Guide for the Care and Use of Laboratory Animals published by the US National Institutes of Health (NIH Pub. No. 85-23, 1996). C57BL6 mice (weight between 25-30 g and 12-14 weeks old) were obtained from Jackson Laboratories for our studies. Mice were kept in an animal care facility for a week prior to the studies. All mice were fed a normal chow diet (Teklad Global diets, Harlan Laboratories).

### Reagents

The primary antibodies used were Bcl-2, Beclin-1, PPAR-γ, p-AKT, total-AKT, p-GSK-3β, total-GSK-3β (Cell Signaling, USA); and β-actin (BD Biosciences Pharmingen, USA). The secondary antibodies used were anti-rabbit IRdye800, anti-mouse IRdye700 (1:50,000 dilution). GSK2B1 (3 μM), Rosiglitazone (6 mg/kg body weight) were purchased from Sigma-Aldrich, USA. Phosphatidylinositol 3-kinase (PI3K)/AKT inhibitor LY-294002 (10 μM) was purchased from Calbiochem. The doses of the inhibitors and agonist used in this study were based on publications in the literature¹.

The omega-3 fish oil-based emulsion used (10 g of TG/100 mL) was a commercially prepared intravenous phospholipid-stabilized emulsion, as described in Example 5 and Table 1 (10% omega-3 oil by weight in grams per 100 ml of emulsion; >90% triglyceride [TG] by weight per total weight of the omega-3 oil, and in which up to about 30% of the total acyl groups are DHA), herein n-3TG or n-3TG90-DHA30. This n-3 TG buffer/emulsion was administered in vivo following H/I to reduce reperfusion injury at a concentration of 10% omega-3 oil by weight in grams per 100 ml of emulsion. For the ex vivo reperfusion experiments, 300 mg of the n-3 TG emulsion was added to a final volume of 100 ml Krebs-buffer for a 0.3% n-3 TG emulsion.

### In Vivo Left Anterior Descending Coronary Artery (LAD) Occlusion

In vivo murine model of ischemia-reperfusion injury: Prior to surgery, mice were anesthetized with isoflurane inhalation (4% induction followed y 1-2.5% maintenance). Subsequent to anaesthesia, mice were orally intubated with polyethylene-60 (PE-60) tubing, connected to a mouse ventilator (MiniVent Type 845, Hugo-Sachs Elektronik) set at a tidal volume of 240 μL and a rate of 110 breaths per minute, and supplemented with oxygen. Body temperature was maintained at 37° C. A median sternotomy was performed, and the proximal left coronary artery (LAD) was visualized and ligated with 7-0 silk suture mounted on a tapered needle (BV-1, Ethicon). After 30 min of ischemia, the prolene suture was cut and the LAD blood flow was restored. Immediately after, intraperitoneal (IP) injection of n-3 TG emulsion (1.5 g/kg body weight) was performed and the second injection was done after 60 min of reperfusion. Control animals received IP injection of saline solution following the same time course. The chest wall was closed, and mice were treated with buprenorphine and allowed to recover in a temperature-controlled area^{4,5}.

### Echocardiogram

In vivo transthoracic echocardiography was performed using a Visual Sonics Vevo 2100 ultrasound biomicroscopy system. This high-frequency (40 MHz) ultrasound system has an axial resolution of ˜30-40 microns and a temporal resolution of >100 Hz. Baseline echocardiography images was obtained prior to myocardial ischemia and post-ischemic images were obtained after 48 hours of reperfusion. The mice were lightly anesthetized with isoflurane (1.5-2.0 L/min) in 100% O₂ and in vivo transthoracic echocardiography of the left ventricle (LV) using a MS-400 38-MHz microscan transducer was used to obtain high resolution two dimensional mode images. Images were used to measure LV end-diastolic diameter (LVEDD), LV end-systolic diameter (LVESD), LV ejection fraction (EF) and LV fractional shortening (FS) as published earlier.^{4,5}

### Infarct Size Measurement

Myocardial infarct size determination: At 48 h of reperfusion mice were re-anesthetized, intubated, and ventilated using a mouse ventilator. A catheter (PE-10 tubing) was placed in the common carotid artery to allow for Evans blue dye injection. A median sternotomy was performed and the LAD was re-ligated in the same location as before. Evans blue dye (1.25 ml of a 7.0% solution) was injected via the carotid artery catheter into the heart to delineate the non-ischemic zone from the ischemic zone. The heart was then rapidly excised and fixed in 1.5% agarose. After the gel solidified, the heart was sectioned perpendicular to the long axis in 1-mm sections using a tissue chopper. The 1-mm sections was placed in individual wells of a six-well cell culture plate and counterstained with 1% TTC for 4 min at 37° C. to demarcate the nonviable myocardium. Each of the 1 mm thick myocardial slices was imaged and weighed. Images were captured using a Q-Capture digital camera connected to a computer. Images were analysed using computer-assisted planimetry with NIH Image 1.63 software to measure the areas of infarction, and total risk area.^{4,5}

### Ex Vivo Ischemia and Reperfusion (I/R)

Experiments were carried out and modified for use in mice hearts.^{4,5} C57BL6 mice between 25-30 g in weight and 12-14 weeks old were anesthetized by injecting ketamine/xylazine cocktail (80 mg/kg and 10 mg/kg respectively). The hearts, rapidly excised, were retrograde perfused through the aorta in a non-recirculating mode, using an isovolumic perfusion system through Langendorff technique (LT), with Krebs-Henseleit buffer, containing (in mM) the following: 118 NaCl, 4.7 KCl, 2.5 CaCl₂, 1.2 MgCl₂, 25 NaHCO₃, 5 Glucose, 0.4 Palmitate, 0.4 BSA, and 70 mU/l insulin. Perfusion pO₂>600 mmHg was maintained in the oxygenation chamber.

Left ventricular developed pressure (LVDP) was continuously monitored, using a latex balloon placed on the left ventricle and connected to a pressure transducer (Gould Laboratories; Pasadena, Calif.). Cardiac function measurements were recorded on a 2-channel ADI recorder. The experimental plan included an equilibration baseline period of 30 min normoxic perfusion followed by 30 min global zero-flow ischemia and 60 min of reperfusion. The flow rate was 2.5 ml/min. The perfusion apparatus was tightly temperature controlled for maintaining heart temperature at 37±0.1° C. under all conditions. The control heart received Krebs-Henseleit buffer; and n-3 TG reperfused hearts received the standard Krebs-Henseleit buffer to which 300 mg n-3 TG emulsion was added per 100 ml final volume, thus making a 0.3% n-3 TG emulsion.

### Assay of Lactate Dehydrogenase (LDH)

Myocardial injury was assessed by measuring the release of lactate dehydrogenase (LDH) from the effluent in the ex vivo I/R system and from blood samples in the in vivo LAD system, using the commercially available enzymatic kits (Pointe Scientific, INC, MI USA) as published earlier.^{4,5}

### Western Blot Analysis

The tissue and cell protein concentration was determined using a DC Protein Assay kit (Bio-Rad). Equal amounts of protein were separated by SDS-PAGE (4-12% gradient gels), and proteins were loaded to a nitrocellulose membrane (Invitrogen). After blocking nonspecific binding with the Odyssey blocking buffer (Li-Cor Biosciences), membranes were incubated overnight at 4° C. with target primary antibodies (1:1,000 dilution), according to the manufacturer's instructions. Successively, membranes were incubated with infrared labeled secondary antibodies for 1 h at room temperature. The bound complex was visualized using the Odyssey Infrared Imaging System (Li-Cor; Lincoln, Nebr.). The images were analyzed using the Odyssey Application Software, version 1.2 (Li-Cor) to obtain the integrated intensities.

### Statistical Analysis

Data were expressed as the mean±SD. For assessing the difference between values, the Student's t test was used. A value of p<0.05 was considered statistically significant.

### In Vivo “n-3 TG” Administration Reduced Infarct Size and Improved Cardiac Function in LAD Model.

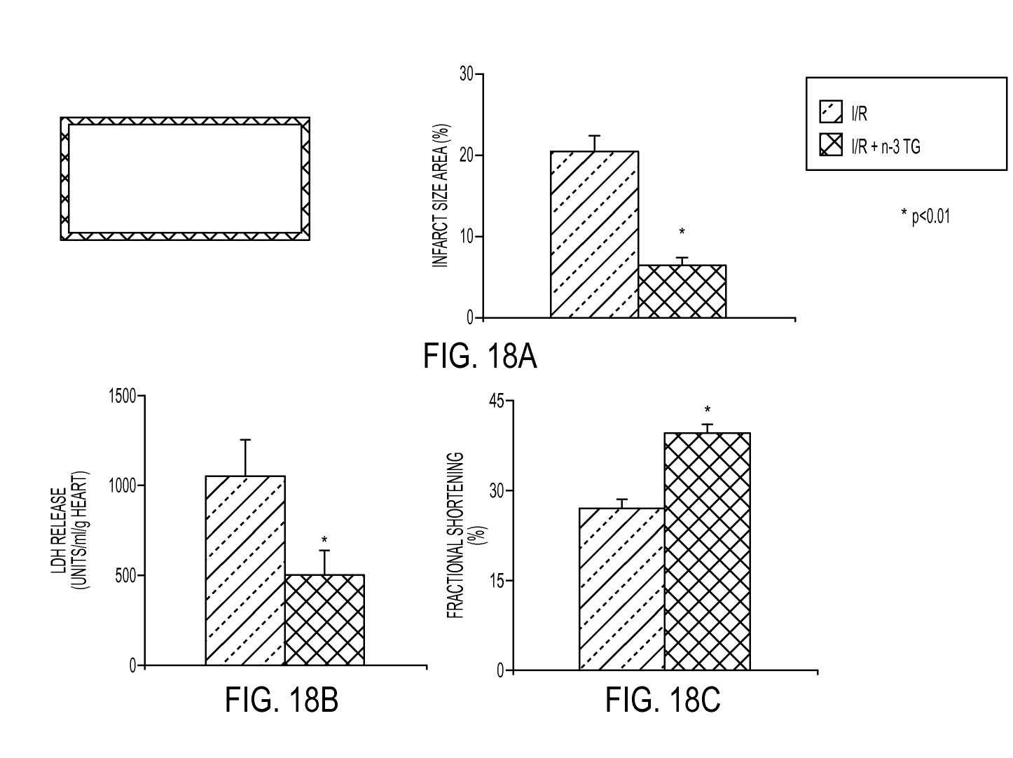

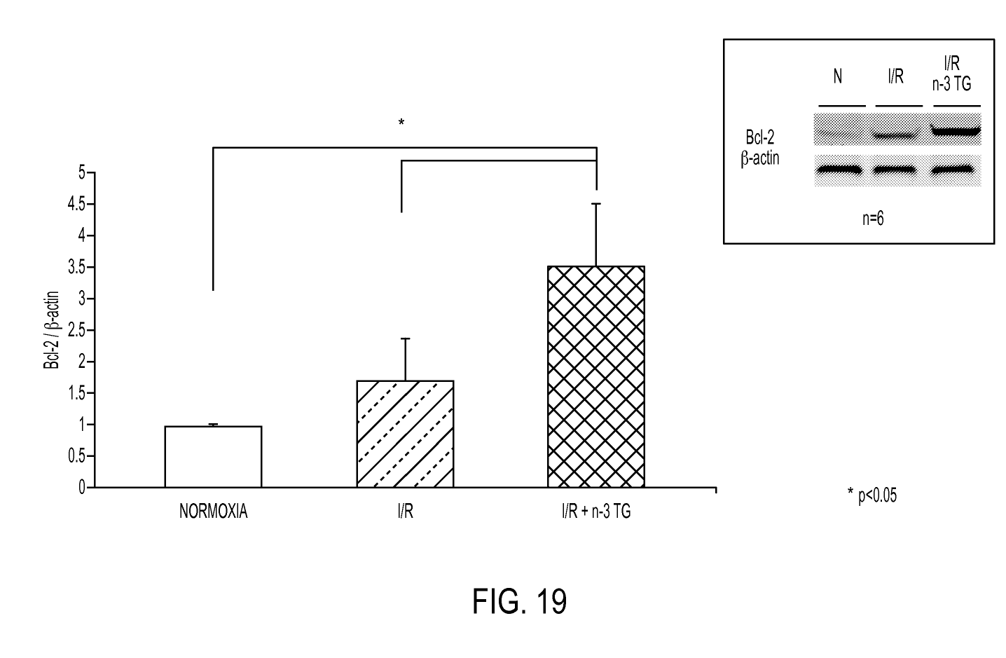

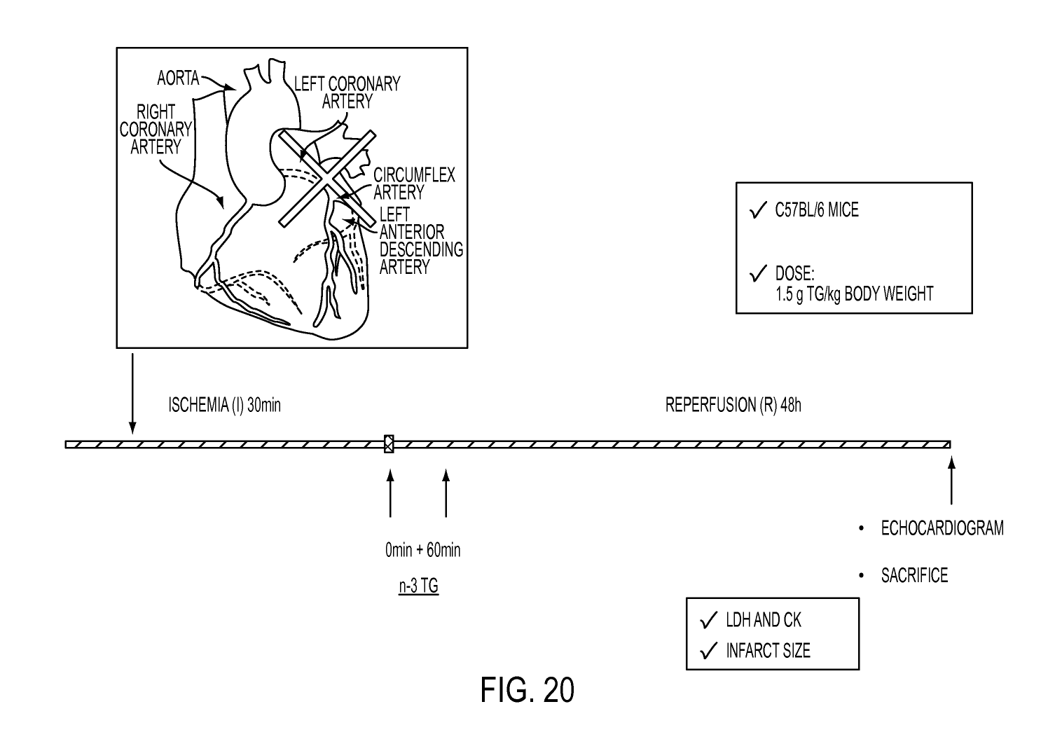

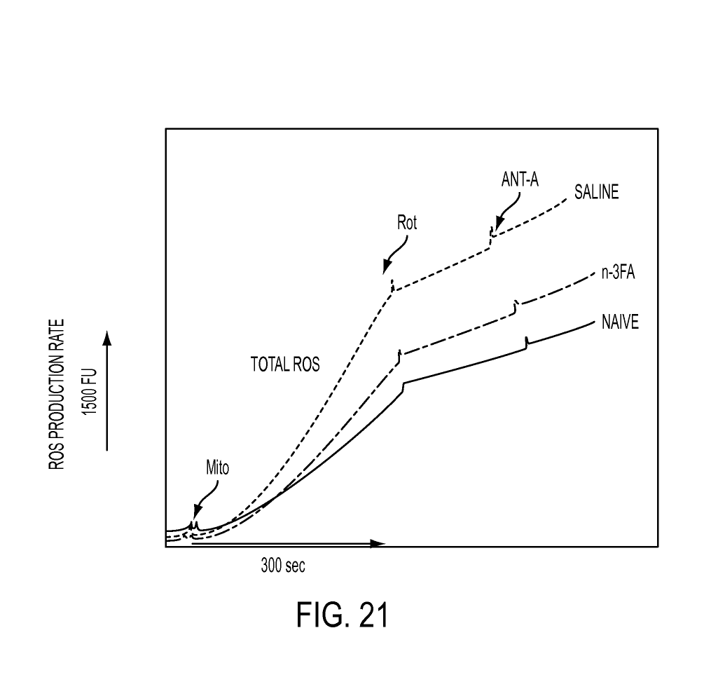

FIG. 18A-18C show that acute i.p. injection of n-3 TG emulsion in vivo decreased infarct size in mouse heart after hypoxic-ischemic injury (FIG. 18A); decreased LDH release which is a marker for heart cell damage (FIG. 18B); and also maintained heart function as shown by the echocardiogram being more normal (FIG. 18C). Bcl-2 (B-cell lymphoma 2), encoded in humans by the BCL2 gene, is the founding member of the Bcl-2 family of regulator proteins that regulate cell death (apoptosis) and it is specifically considered as an important anti-apoptotic protein and is thus classified as an oncogene. An increase in gene expression and mouse heart protein expression of BCL-2 which is an anti-apoptotic marker for (left) normoxia, (middle) control, and (right) after administration of n-3 TG post ischemic event are shown in FIG. 19. Bcl-2 is increased after myocardial infarction in untreated animals, but is increased even more (by up to about 3.5 times) the level observed in normoxic conditions in animals treated with n-3 TG. FIG. 20 illustrates an in vivo left anterior descending coronary artery (LAD) occlusion model. FIG. 21 shows that n-3 TG decreases mitochondrial production of reactive oxidation species (ROS).

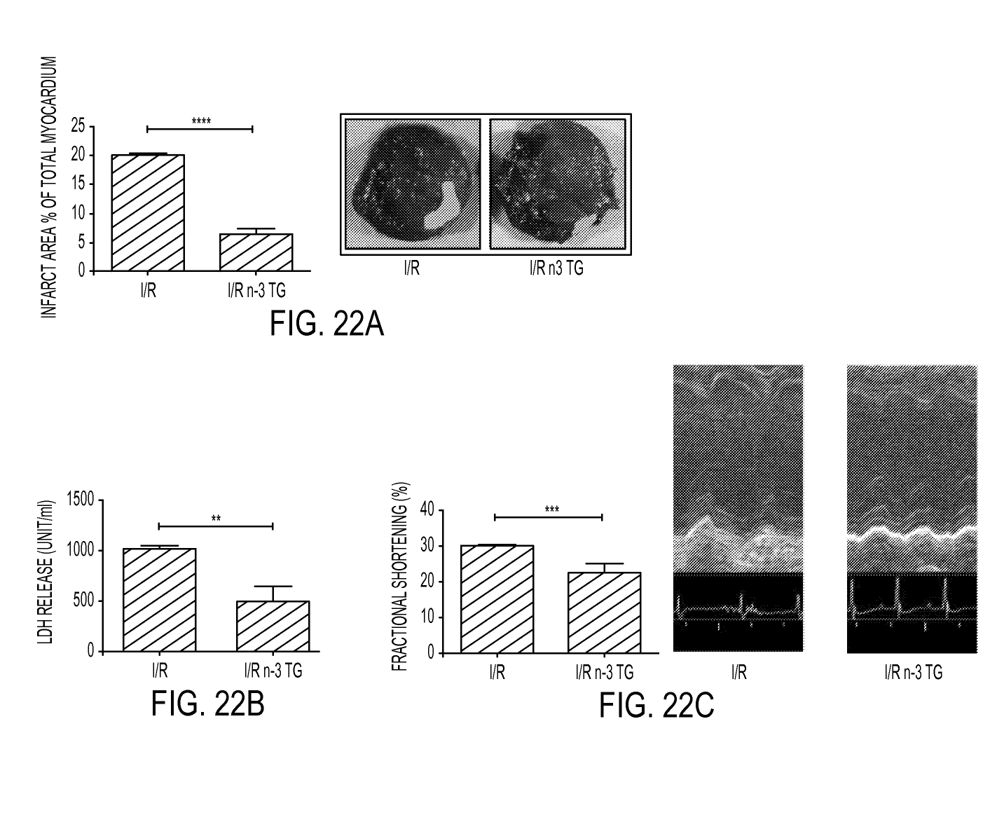

To test the effect of acute n-3 TG administration in myocardial ischemic injury, mice were subjected to 30 min of ischemia induced by LAD occlusion; coronary flow was then restored and myocardial functional recovery during reperfusion was assessed. IP injection of n-3 TG emulsion was administered immediately after ischemia at the onset of reperfusion and at 60 min into reperfusion. At the end of 48 h of reperfusion, sections of heart were stained with TTC to quantify the extent of I/R damage in both groups. FIG. 22A shows quantification of the infarct area in mice hearts from saline treated compared to n-3 TG treated group. Myocardial infarct size was significantly reduced (p<0.05) in n-3 TG emulsion treated mice (vs saline treated mice). The total area at risk was similar for both groups. Plasma LDH release, a key marker of myocardial injury, was significantly reduced in n-3 TG treated mice (FIG. 22B). These data indicate that acute treatment of n-3 TG during reperfusion markedly reduces injury due to myocardial infarction in mice.

Echocardiography assessment showed substantial differences in fractional shortening (% FS) between control and n-3 TG treated mice. A significant recovery of FS was observed in n-3 TG treated group vs saline treated controls (p<0.01) (FIG. 22C). These data along with infarct size changes and LDH levels reduction reveal that acute n-3 TG treatment protects mice from myocardial ischemia-reperfusion injury and improves heart function.

In Ex-Vivo Model n-3 TG Protects Myocardium from I/R Injury

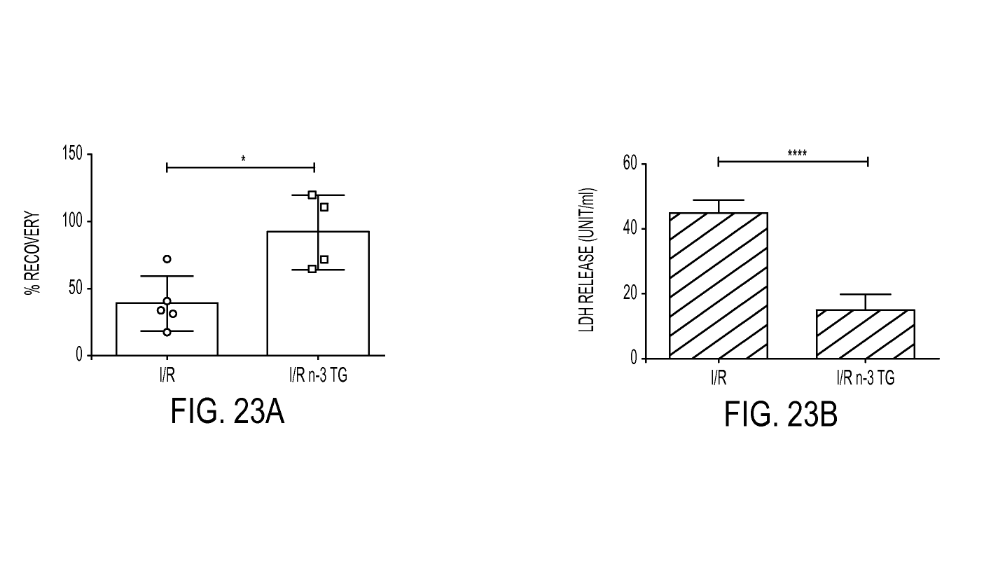

To investigate further the effect of acute intervention with n-3 TG emulsion after I/R, the ex vivo perfused heart was used (I/R model). The experiments showed that administration of a 0.3% n-3 TG emulsion during reperfusion in the ex vivo model significantly improved LVDP recovery after I/R (FIG. 23A), compared to control hearts. Reperfusion of the heart with KREB'S buffer+0.3% n-3 TG maintained normal rhythm and LVDP was nearly restored to 100% similar to pre-ischemia time.

During reperfusion period, heart perfusates were collected to detect LDH release, as markers of ischemic injury. LDH release appeared significant different between 0.3% n-3 TG treated and control hearts, showing that acute n-3 TG treatment exhibits a protective role (FIG. 23B).

### N-3 TG Modulates Key Signalling Pathways Linked to I/R Injury.

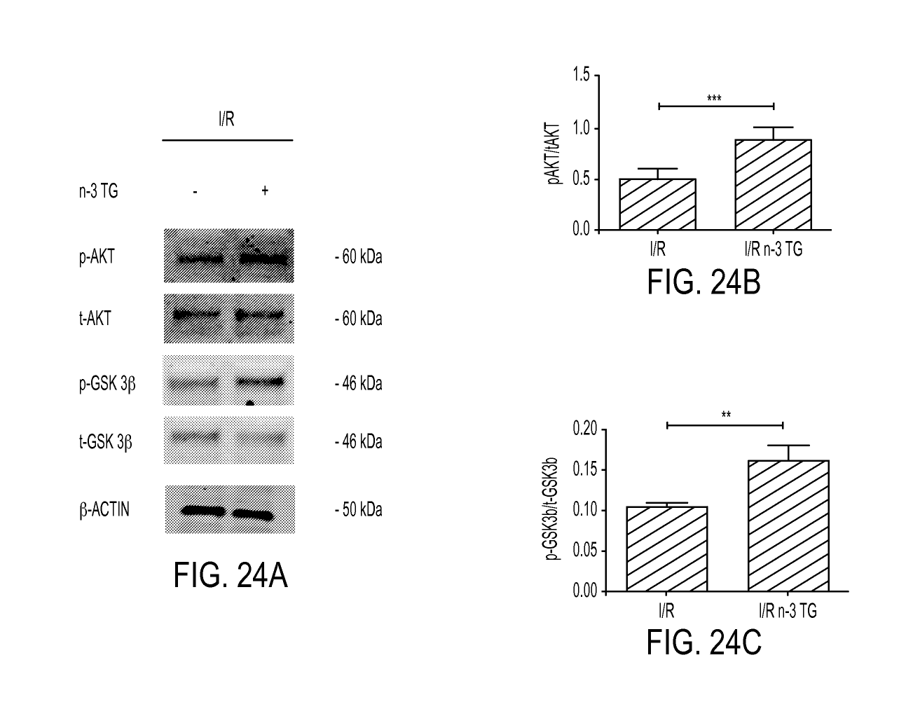

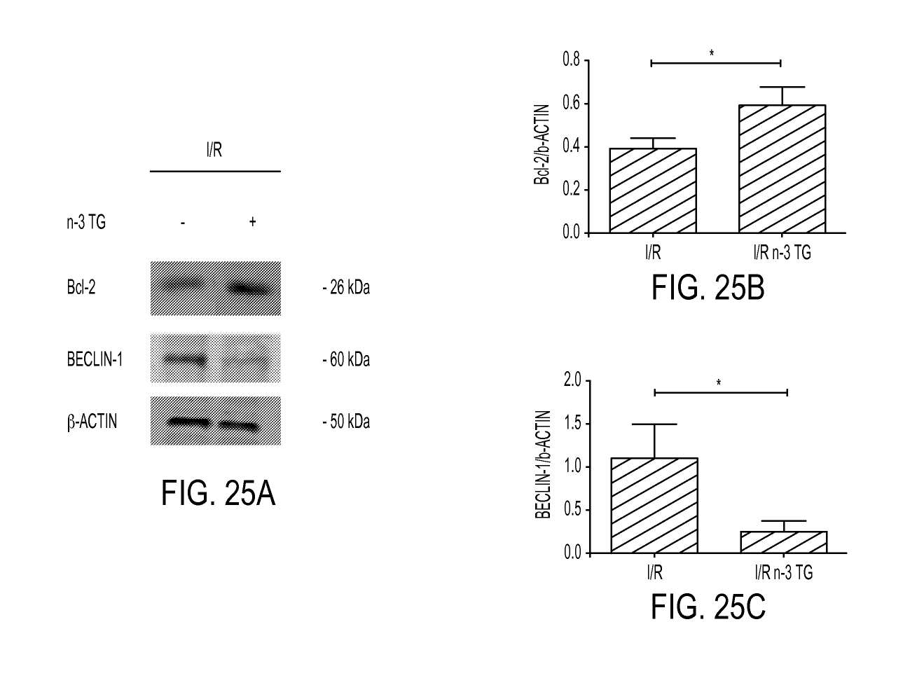

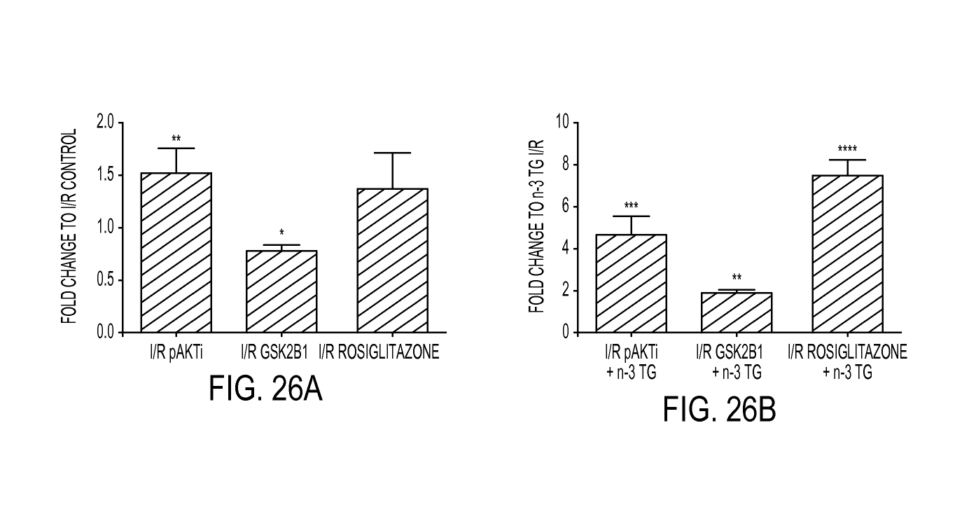

To determine if omega-3 triglyceride protects hearts by modulating changes in key signalling pathways linked to I/R injury, p-AKT, p-GSK-3β, and Bcl-2 were probed in myocardial tissue by western blotting (FIG. 24A). Reperfusion ex vivo in 0.3% n-3 TG emulsion significantly increased phosphorylation of AKT (FIG. 24B) and GSK3β (FIG. 24C), and Bcl-2 protein expression (FIG. 25), indicating that n-3 TG likely reduces apoptosis by activating the PI3K-AKT-GSK3β signalling pathway and anti-apoptotic protein Bcl-2. Since Bcl-2 interacts with Beclin-1^{6,7} and influences autophagy, as shown in FIG. 25A-25C, the expression of Beclin-1 increased after ischemia/reperfusion condition; 0.3% n-3 TG-treated hearts showed a significant reduction in Beclin-1 protein expression, with concomitant increase of Bcl-2 protein expression as mentioned above. To establish the link between n-3 TG and PI3K/AKT and GSK3β pathways in I/R injury, hearts were treated with GSK-3β inhibitor GSK2B1 (3 μM) or Phosphatidylinositol 3-kinase (PI3K)/AKT inhibitor LY-294002 (10 μM); each of them was added at the beginning of the baseline period and continued throughout ischemia and reperfusion. The doses of the inhibitors used in this study were based on publications in the literature¹. LDH release was significantly reduced by n-3 TG, and protection afforded by n-3 TG was abrogated PI3K/AKT inhibitor, LY-294002 (FIG. 26A-26B). Treatment with GSK2B1 plus n-3 TG emulsion significantly inhibited LDH release compared to control hearts (FIG. 26A-26B).

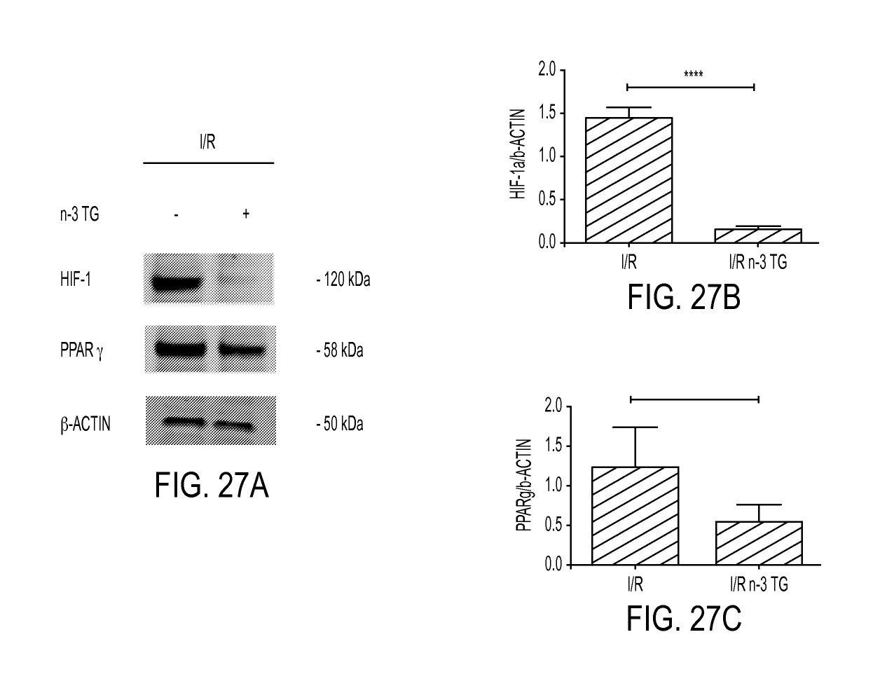

Next, hypoxia-inducible factor 1 (HIF-1) was investigated, which is a key mediator of adaptive responses to decreased oxygen availability in ischemia. HIF-1 protein expression (FIG. 27A-27B) increased rapidly after ischemia. Administration of even as little as 0.3% n-3 TG during reperfusion significantly inhibited the protein expression of HIF-1α. In our lab, previous studies showed that n-3 fatty acids, in contrast to saturated fatty acids, are able to lower macrophages and arterial endothelial lipase and inflammatory markers and these effects are linked to PPAR-γ⁸. Accordingly, the potential association of PPAR-γ and n-3 TG acute treatment in I/R condition was examined. (FIG. 27C). Western blot analysis showed that in n-3 TG treated hearts protein expression of PPAR-γ was significantly lower compared to the control hearts (FIG. 27A).

In order to establish the link between PPAR-γ and n-3 TG effect, mice were treated with Rosiglitazone (6 mg/kg body weight, IP injection), a common agonist of PPAR-γ, 30 min before I/R injury in the isolated perfused hearts. These hearts were perfused with Krebs-Henseleit buffer without or with n-3 TG (300 mg/100 ml buffer) emulsion during reperfusion time. LDH release was significantly higher in Rosiglitazone plus n-3 TG treated hearts vs Rosiglitazone treated hearts (FIG. 26A-26B). These data indicate that PPAR-γ reduction is linked to cardioprotection afforded by n-3 TG during I/R.

Taken together, these results suggest that PI3K/AKT, GSK-3β and PPAR-γ are key pathways modulating n-3 TG cardioprotection.

### REFERENCES CITED IN EXAMPLE 8

Abdillahi M, Ananthakrishnan R, Vedantham S, Shang L, Zhu Z, Rosario R, Zirpoli H, Bohren K M, Gabbay K H, Ramasamy R. (2012) Aldose reductase modulates cardiac glycogen synthase kinase-3β phosphorylation during ischemia-reperfusion. Am J Physiol Heart Circ Physiol. 303(3): H297-308.
Ananthakrishnan, R., et al., Aldose reductase mediates myocardial ischemia-reperfusion injury in part by opening mitochondrial permeability transition pore. Am J Physiol Heart Circ Physiol, 2009. 296(2): p. H333-41.
Bellot G, Garcia-Medina R, Gounon P, Chiche J, Roux D, Pouysségur J, Mazure N M. (2009) Hypoxia-induced autophagy is mediated through hypoxia-inducible factor induction of BNIP3 and BNIP3L via their BH3 domains. Mol Cell Biol. 29(10):2570-81.

Hwang, Y. C., et al., Central role for aldose reductase pathway in myocardial ischemic injury. FASEB J, 2004. 18(11): p. 1192-9.

Qi K, Seo T, Al-Haideri M, Worgall T S, Vogel T, Carpentier Y A, Deckelbaum R J. (2002) Omega-3 triglycerides modify blood clearance and tissue targeting pathways of lipid emulsions. Biochemistry. 41(9): 3119-27.
Rauch B, Schiele R, Schneider S, Diller F, Victor N, Gohlke H, Gottwik M, Steinbeck G, Del Castillo U, Sack R, Worth H, Katus H, Spitzer W, Sabin G, Senges J; OMEGA Study Group. (2010) OMEGA, a randomized, placebo-controlled trial to test the effect of highly purified omega-3 fatty acids on top of modern guideline-adjusted therapy after myocardial infarction. Circulation. 122(21): 2152-9.
Vacek T P, Vacek J C, Tyagi N, Tyagi S C. (2012) Autophagy and heart failure: a possible role for homocysteine. Cell Biochem Biophys. 62(1):1-11.
Williams J J, Mayurasakorn K, Vannucci S J, Mastropietro C, Bazan N G, Ten V S, Deckelbaum R J. (2013) N-3 fatty acid rich triglyceride emulsions are neuroprotective after cerebral hypoxic-ischemic injury in neonatal mice. PLoS One; 8(2):e56233.

### REFERENCES

1. Lloyd-Jones D, Adams R J, Brown T M, Carnethon M, Dai S, et al. (2009) Heart disease and stroke statistics-2010 update: a report from the American Heart Association. Circulation 121: e46-e215.
2. Raju T N, Nelson K B, Ferriero D, Lynch J K (2007) Ischemic perinatal stroke: summary of a workshop sponsored by the National Institute of Child Health and Human Development and the National Institute of Neurological Disorders and Stroke. Pediatrics 120: 609-616.
3. Callaway J K (2001) Investigation of AM-36: a novel neuroprotective agent. Clin Exp Pharmacol Physiol 28: 913-918.
4. Legido A, Christos K (2000) Perinatal Hypoxic Ischemic Encephalopathy: Current and Future Treatments. International Pediatrics 15: 143-151.
5. Mayurasakorn K, Williams J J, Ten V S, Deckelbaum R J (2011) Docosahexaenoic acid: brain accretion and roles in neuroprotection after brain hypoxia and ischemia. Curr Opin Clin Nutr Metab Care 14: 158-167.
6. Calder P C (2010) Omega-3 fatty acids and inflammatory processes. Nutrients 2: 355-374.
7. Deckelbaum R J, Worgall T S, Seo T (2006) n-3 fatty acids and gene expression. Am J Clin Nutr 83: 1520S-1525S.
8. Seo T, Blaner W S, Deckelbaum R J (2005) Omega-3 fatty acids: molecular approaches to optimal biological outcomes. Curr Opin Lipidol 16: 11-18.
9. Bazan N G, Molina M F, Gordon W C (2011) Docosahexaenoic acid signalolipidomics in nutrition: significance in aging, neuroinflammation, macular degeneration, Alzheimer's, and other neurodegenerative diseases. Annu Rev Nutr 31: 321-351.
10. Perlman J M (2007) Pathogenesis of hypoxic-ischemic brain injury. Journal of Perinatology 27: S39-S46.
11. Belayev L, Marcheselli V L, Khoutorova L, Rodriguez de Turco E B, Busto R, et al. (2005) Docosahexaenoic acid complexed to albumin elicits high-grade ischemic neuroprotection. Stroke 36: 118-123.
12. Singh A K, Yoshida Y, Garvin A J, Singh I (1989) Effect of fatty acids and their derivatives on mitochondrial structures. J Exp Pathol 4: 9-15.
13. Oliveira F L, Rumsey S C, Schlotzer E, Hansen I, Carpentier Y A, et al. (1997) Triglyceride hydrolysis of soy oil vs fish oil emulsions. JPEN J Parenter Enteral Nutr 21: 224-229.
14. Adolph M (1999) Lipid emulsions in parenteral nutrition. Ann Nutr Metab 43: 1-13.
15. Vannucci S J, Hagberg H (2004) Hypoxia-ischemia in the immature brain. J Exp Biol 207: 3149-3154.
16. Pavlakis S G, Hirtz D G, DeVeber G (2006) Pediatric stroke: Opportunities and challenges in planning clinical trials. Pediatr Neurol 34: 433-435.
17. Ten V S, Bradley-Moore M, Gingrich J A, Stark R I, Pinsky D J (2003) Brain injury and neurofunctional deficit in neonatal mice with hypoxic-ischemic encephalopathy. Behav Brain Res 145: 209-219.
18. Qi K, Seo T, Al-Haideri M, Worgall T S, Vogel T, et al. (2002) Omega-3 triglycerides modify blood clearance and tissue targeting pathways of lipid emulsions. Biochemistry 41: 3119-3127.
19. Qi K, Al-Haideri M, Seo T, Carpentier Y A, Deckelbaum R J (2003) Effects of particle size on blood clearance and tissue uptake of lipid emulsions with different triglyceride compositions. JPEN J Parenter Enteral Nutr 27: 58-64.
20. Granot E, Schwiegelshohn B, Tabas I, Gorecki M, Vogel T, et al. (1994) Effects of particle size on cell uptake of model triglyceride-rich particles with and without apoprotein E. Biochemistry 33: 15190-15197.
21. Schwiegelshohn B, Presley J F, Gorecki M, Vogel T, Carpentier Y A, et al. (1995) Effects of apoprotein E on intracellular metabolism of model triglyceride-rich particles are distinct from effects on cell particle uptake. J Biol Chem 270: 1761-1769.
22. Liszczak T M, Hedley-Whyte E T, Adams J F, Han D H, Kolluri V S, et al. (1984) Limitations of tetrazolium salts in delineating infarcted brain. Acta Neuropathol 65: 150-157.
23. Denis C, Methia N, Frenette P S, Rayburn H, Ullman-Cullere M, et al. (1998) A mouse model of severe von Willebrand disease: defects in hemostasis and thrombosis. Proc Natl Acad Sci USA 95: 9524-9529.
24. Niatsetskaya Z V, Sosunov S A, Matsiukevich D, Utkina-Sosunova I V, Ratner V I, et al. (2012) The oxygen free radicals originating from mitochondrial complex I contribute to oxidative brain injury following hypoxia-ischemia in neonatal mice. J Neurosci 32: 3235-3244.
25. Bruno A, Biller J, Adams H P, Jr., Clarke W R, Woolson R F, et al. (1999) Acute blood glucose level and outcome from ischemic stroke. Trial of ORG 10172 in Acute Stroke Treatment (TOAST) Investigators. Neurology 52: 280-284.
26. Caspersen C S, Sosunov A, Utkina-Sosunova I, Ratner V I, Starkov A A, et al. (2008) An isolation method for assessment of brain mitochondria function in neonatal mice with hypoxic-ischemic brain injury. Dev Neurosci 30: 319-324.
27. Berman D R, Mozurkewich E, Liu Y, Barks J (2009) Docosahexaenoic acid pretreatment confers neuroprotection in a rat model of perinatal cerebral hypoxia-ischemia. Am J Obstet Gynecol 200: 305 e301-306.
28. Williams J J, Bazan N G, Ten V S, Vannucci S J, Mastropietro C, et al. (2009) n-3 fatty acids are neuroprotective after cerebral hypoxia-ischemia in rodent models. FASEB J 23: 334.335 (Abstract).
29. Qi K, Seo T, Jiang Z, Carpentier Y A, Deckelbaum R J (2006) Triglycerides in fish oil affect the blood clearance of lipid emulsions containing long- and medium-chain triglycerides in mice. J Nutr 136: 2766-2772.
30. Pan H C, Kao T K, Ou Y C, Yang D Y, Yen Y J, et al. (2009) Protective effect of docosahexaenoic acid against brain injury in ischemic rats. J Nutr Biochem 20: 715-725.
31. Huang J, Mocco J, Choudhri T F, Poisik A, Popilskis S J, et al. (2000) A modified transorbital baboon model of reperfused stroke. Stroke 31: 3054-3063.
32. Mayurasakorn K, Williams J J, Ten V S, Deckelbaum R J (2011) n-3 but not n-6 fatty acids prevent brain death after hypoxic ischemic injury. FASEB J 25 777.710 (Abstract).
33. Grimsgaard S, Bonaa K H, Hansen J B, Nordoy A (1997) Highly purified eicosapentaenoic acid and docosahexaenoic acid in humans have similar triacylglycerol-lowering effects but divergent effects on serum fatty acids. Am J Clin Nutr 66: 649-659.
34. Stanley W C, Khairallah R J, Dabkowski E R (2012) Update on lipids and mitochondrial function: impact of dietary n-3 polyunsaturated fatty acids. Curr Opin Clin Nutr Metab Care 15: 122-126.
35. Khairallah R J, Sparagna G C, Khanna N, O'Shea K M, Hecker P A, et al. (2010) Dietary supplementation with docosahexaenoic acid, but not eicosapentaenoic acid, dramatically alters cardiac mitochondrial phospholipid fatty acid composition and prevents permeability transition. Biochim Biophys Acta 1797: 1555-1562.
36. Belayev L, Khoutorova L, Atkins K D, Bazan N G (2009) Robust docosahexaenoic acid-mediated neuroprotection in a rat model of transient, focal cerebral ischemia. Stroke 40: 3121-3126.
37. Bazan N G (2012) Neuroinflammation and Proteostasis are Modulated by Endogenously Biosynthesized Neuroprotectin D1. Mol Neurobiol 46: 221-226.
38. Marcheselli V L, Hong S, Lukiw W J, Tian X H, Gronert K, et al. (2003) Novel docosanoids inhibit brain ischemia-reperfusion-mediated leukocyte infiltration and pro-inflammatory gene expression. J Biol Chem 278: 43807-43817.
39. Mukherjee P K, Marcheselli V L, Serhan C N, Bazan N G (2004) Neuroprotectin D1: a docosahexaenoic acid-derived docosatriene protects human retinal pigment epithelial cells from oxidative stress. Proc Natl Acad Sci USA 101: 8491-8496.
40. Belayev L, Khoutorova L, Atkins K D, Eady T N, Hong S, et al. (2011) Docosahexaenoic Acid Therapy of Experimental Ischemic Stroke. Transl Stroke Res 2: 33-41.
41. Terpstra A H (2001) Differences between humans and mice in efficacy of the body fat lowering effect of conjugated linoleic acid: role of metabolic rate. J Nutr 131: 2067-2068.
42. Blaxter K (1989) Energy metabolism in animals and man: Cambridge University Press, Cambridge, UK.
43. Miller A T (1986) Energy Metabolism. F.A. Davis Company, Philadelphia, Pa.
44. Lewandowski C, Barsan W (2001) Treatment of acute ischemic stroke. Ann Emerg Med 37: 202-216.
45. Blondeau N, Petrault O, Manta S, Giordanengo V, Gounon P, et al. (2007) Polyunsaturated fatty acids are cerebral vasodilators via the TREK-1 potassium channel. Circ Res 101: 176-184.
46. NINDS (1995) Tissue plasminogen activator for acute ischemic stroke. The National Institute of Neurological Disorders and Stroke rt-PA Stroke Study Group. N Engl J Med 333: 1581-1587.
47. NINDS (1997) Generalized efficacy of t-PA for acute stroke. Subgroup analysis of the NINDS t-PA Stroke Trial. Stroke 28: 2119-2125.
48. Dirnagl U, Iadecola C, Moskowitz M A (1999) Pathobiology of ischaemic stroke: an integrated view. Trends Neurosci 22: 391-397.
49. Ten V S, Starkov A (2012) Hypoxic-ischemic injury in the developing brain: the role of reactive oxygen species originating in mitochondria. Neurol Res Int 2012: 542976.
50. Williams J J, Deckelbaum R J, Mayurasakorn K, Ten V S (2011) n-3 Fatty acids protect brain against hypoxia-ischemia (HI) by attenuation of oxidative injury and mitochondrial dysfunction FASEB J 25: 105.104 (Abstract).
51. Lim S N, Huang W, Hall J C, Ward R E, Priestley J V, et al. (2010) The acute administration of eicosapentaenoic acid is neuroprotective after spinal cord compression injury in rats. Prostaglandins Leukot Essent Fatty Acids 83: 193-201.
52. Ward R E, Huang W, Curran O E, Priestley J V, Michael-Titus A T (2010) Docosahexaenoic acid prevents white matter damage after spinal cord injury. J Neurotrauma 27: 1769-1780.
53. King V R, Huang W L, Dyall S C, Curran O E, Priestley J V, et al. (2006) Omega-3 fatty acids improve recovery, whereas omega-6 fatty acids worsen outcome, after spinal cord injury in the adult rat. J Neurosci 26: 4672-4680.
54. Williams J J, Mayurasakorn K, Vannucci S J, Mastropietro C, Bazan N G, et al. (2013) N-3 Fatty Acid Rich Triglyceride Emulsions Are Neuroprotective after Cerebral Hypoxic-ischemic Injury in Neonatal Mice. PLoS ONE 8(2): e56233. doi:10.1371/journal.pone.0056233
55. Racine R A, Deckelbaum, R J. (2007) Sources of the very-long-chain unsaturated omega-3 fatty acids: eicosapentaenoic acid and docosahexaenoic acid. Current Opinion in Clinical Nutrition and Metabolic Care 2007, 10:123-128.

What is claimed is:

1. A triglyceride omega-3 lipid-based oil-in-water emulsion suitable for administration to a patient, wherein
(a) the emulsion comprises at least 7% to 35% omega-3 oil and less than 10% omega-6 oil by weight in grams per 100 ml of emulsion,
(b) the omega-3 oil comprises at least 20% triglyceride by weight per total weight of the omega-3 oil, and at least 20% wt.-% of the acyl-groups of the omega-3 triglycerides consist of DHA,
(c) the omega-3 oil comprises less than 10% omega-6 fatty acids, and
(d) the mean diameter of lipid droplets in the emulsion is less than about 5 microns.

2. The triglyceride omega-3 lipid-based oil-in-water emulsion of claim 1, wherein the omega-3 oil is fish oil, synthetic omega-3 oil or a combination thereof.

3. The triglyceride omega-3 lipid-based oil-in-water emulsion of claim 1, wherein from 20 wt.-% to 50 wt.-% of the acyl-groups of the omega-3 triglycerides consist of DHA.

4. The triglyceride omega-3 lipid-based oil-in-water emulsion of claim 1, wherein from 50 wt.-% to 75 wt.-% of the acyl-groups of the omega-3 triglycerides consist of DHA.

5. The triglyceride omega-3 lipid-based oil-in-water emulsion of claim 1, wherein from 75 to 90 wt.-% of the acyl-groups of the omega-3 triglycerides consist of DHA.

6. The triglyceride omega-3 lipid-based oil-in-water emulsion of claim 1, wherein from 90 to 95 wt.-% of the acyl-groups of the omega-3 triglycerides consist of DHA.

7. The triglyceride omega-3 lipid-based oil-in-water emulsion of claim 1, wherein from 95 to 100 wt.-% of the acyl-groups of the omega-3 triglycerides consist of DHA.

8. The triglyceride omega-3 lipid-based oil-in-water emulsion of claim 1, wherein the lipid droplets are less than about 1 micron diameter.

9. The triglyceride omega-3 lipid-based oil-in-water emulsion of claim 1, wherein the lipid droplets are from about 100 to about 500 nm diameter.

10. A method comprising
(a) identifying a subject who has undergone hypoxia-ischemia or is at risk of having hypoxia/ischemia, and
(b) administering to the subject a pharmaceutical composition comprising a therapeutically effective amount the triglyceride omega-3 lipid-based oil-in-water emulsion of claim 1 to reduce reperfusion damage caused by the hypoxia-ischemia.

11. The method of claim 10, wherein the hypoxia-ischemia causes cerebral hypoxia-ischemia, and the therapeutically effective amount of the triglyceride omega-3 lipid-based oil-in-water emulsion of claim 1 is administered within 20 minutes to 2 hours after the cerebral hypoxia-ischemia to reperfusion damage caused by the hypoxia-ischemia.

12. The method of claim 10, wherein the hypoxia-ischemia causes cerebral hypoxia-ischemia, and the therapeutically effective amount of the triglyceride omega-3 lipid-based oil-in-water emulsion of claim 1 is administered within 2 to 4 hours or 4 to 6 hours after the cerebral hypoxia-ischemia to reperfusion damage caused by the hypoxia-ischemia.

13. The method of claim 10, wherein the cerebral hypoxia-ischemia causes a stroke.

14. The method of claim 10, wherein the therapeutically effective amount is from about 0.05 g/kg/administration to about 4 g/kg/administration.

15. The method of claim 10, wherein the hypoxia-ischemia and the reperfusion damage caused by the hypoxia-ischemia occurs in an organ selected from the group consisting of brain, heart, kidney, spinal cord, large or small intestine, pancreas and lung.

16. The method of claim 10, wherein hypoxia-ischemia and the reperfusion damage caused by the hypoxia-ischemia causes a myocardial infarction or a cerebral infarction.

17. A method for reducing cell death or damage in an organ or tissue caused by hypoxia-ischemia or reperfusion damage caused by the hypoxia-ischemia comprising administering to a patient in need thereof a pharmaceutical composition comprising a therapeutically effective amount the triglyceride omega-3 lipid-based oil-in-water emulsion of claim 1.

18. The method of claim 17, wherein the hypoxia-ischemia or reperfusion damage caused by the hypoxia-ischemia is caused by organ transplantation.

19. The method of claim 17, wherein the cell death or cell damage occurs in an organ selected from the group consisting of brain, heart, kidney, spinal cord, large or small intestine, lung, liver, and pancreas.

20. The method of claim 17, wherein the hypoxia-ischemia causes a stroke.

21. The method of claim 17, wherein the hypoxia-ischemia causes a myocardial infarction.

22. The method of claim 17, wherein the therapeutically effective amount is from about 0.05 g/kg/administration to about 4 g/kg/administration.

23. A method comprising:
(a) identifying a subject who is at risk of having a cerebral hypoxia-ischemia, and
(b) administering to the subject a pharmaceutical composition comprising a therapeutically effective amount of the triglyceride omega-3 lipid-based oil-in-water emulsion of claim 1, thereby reducing the risk of the subject developing reperfusion damage caused by the hypoxia-ischemia.

24. A method comprising:
(a) identifying a subject having inflammation in an organ, and
(b) administering to the subject a pharmaceutical composition comprising a therapeutically effective amount of the triglyceride omega-3 lipid-based oil-in-water emulsion of claim 1, thereby reducing the inflammation in the subject.

25. A method comprising administering to a subject an amount of a pharmaceutical composition comprising the triglyceride omega-3 lipid-based oil-in-water emulsion of claim 1, to reduce production of reactive oxygen species in the blood or in an organ in the subject.

26. The method of claim 25, wherein the organ is selected from the group consisting of brain, heart, kidney, spinal cord, large or small intestine, lung, liver, and pancreas.

27. A method for reducing adverse cytokine production in a subject, comprising administering to a subject in need thereof a therapeutically effective amount of a pharmaceutical composition comprising the triglyceride omega-3 lipid-based oil-in-water emulsion of claim 1.

28. The emulsion of claim 1, wherein the emulsion comprises at least 20% to 30% omega-3 oil by weight in grams per 100 ml of emulsion.

29. The emulsion of claim 1, wherein the emulsion comprises at least 30% to 35% omega-3 oil by weight in grams per 100 ml of emulsion.

30. The emulsion of claim 1, wherein the omega-3 oil comprises at least 30% to 40% triglyceride by weight per total weight of the omega-3 oil.

31. The emulsion of claim 1, wherein the omega-3 oil comprises at least 40% to 50% triglyceride by weight per total weight of the omega-3 oil.

32. The emulsion of claim 1, wherein the omega-3 oil comprises at least 50% to 75% triglyceride by weight per total weight of the omega-3 oil.

33. The emulsion of claim 1, wherein the omega 3 oil comprises at least 75% to 100% triglyceride by weight per total weight of the omega-3 oil.

34. The triglyceride omega-3 lipid-based oil-in-water emulsion of claim 1, wherein the emulsion comprises less than 5% omega-6 fatty acid.

35. The triglyceride omega-3 lipid-based oil-in-water emulsion of claim 1, wherein the emulsion comprises less than 1% omega-6 fatty acid.

36. The triglyceride omega-3 lipid-based oil-in-water emulsion of claim 1, wherein the emulsion comprises at least 35% omega-3 oil by weight in grams per 100 ml of emulsion and less than 3% omega-6 oil.

37. A method comprising
(a) identifying a subject having inflammation in an organ or a tissue, and
(b) administering to the subject a pharmaceutical composition comprising a therapeutically effective amount of the triglyceride omega-3 triglyceride oil-in-water emulsion of claim 1, thereby reducing the inflammation in the subject.

38. A method for reducing cell death or damage or hypoxia/ischemia in an organ or tissue comprising administering to an organ donor a pharmaceutical composition comprising a therapeutically effective amount the triglyceride omega-3 triglyceride oil-in-water emulsion of claim 1 prior to harvesting the organ or tissue from the organ donor.

39. A method for reducing cell death or damage or hypoxia/ischemia in an organ or tissue to be transplanted into an organ or tissue recipient, comprising administering to the recipient a pharmaceutical composition comprising a therapeutically effective amount the triglyceride omega-3 triglyceride oil-in-water emulsion of claim 1 prior to surgically implanting the organ or tissue in the recipient.
